(19) United States
(12) Patent Application Publication
(10) Pub. No.: US 2013/0302794 A1
(43) Pub. Date: Nov. 14, 2013

(54) NUCLEIC ACID DETECTION BY OLIGONUCLEOTIDE PROBES CLEAVED BY BOTH EXONUCLEASE AND ENDONUCLEASE

(71) Applicants: Jun LI, Woodstock, MD (US); Win Den CHEUNG, Olney, MD (US); Jason OPDYKE, Silver Spring, MD (US); John HARVEY, Elkridge, MD (US); Songchun CHONG, Ellicott City, MD (US)

(72) Inventors: Jun LI, Woodstock, MD (US); Win Den CHEUNG, Olney, MD (US); Jason OPDYKE, Silver Spring, MD (US); John HARVEY, Elkridge, MD (US); Songchun CHONG, Ellicott City, MD (US)

(73) Assignee: SAMSUNG TECHWIN CO., LTD., Changwon-city (KR)

(21) Appl. No.: 13/467,586

(22) Filed: May 9, 2012

(51) Int. Cl. C12Q 1/68

(52) U.S. Cl. 435/6.11

(57) ABSTRACT

Disclosed is a method in the fields of biochemistry and molecular biology. The method is related to improve cleavage kinetics of labeled oligonucleotide probes and, consequently, increases signal-to-noise ratio in detecting nucleic acids.

### TECHNICAL FIELD

One or more embodiments related to a method in the fields of biochemistry and molecular biology. The method according to one or more embodiments is related to improve cleavage kinetics of labeled oligonucleotide probes and, consequently, increases the signal-to-noise ratio in detecting nucleic acids.

### BACKGROUND

Most real-time PCR methods including, e.g., the TaqMan® assay, require hybridization and cleavage of oligonucleotide probes. In these types of assays, a detectable probe, which is complementary to the target sequence is added to a PCR reaction mixture. The probe is labeled with a detectable marker, for example a fluorescent reporter dye at one end and a fluorescent quencher at another end. When the probe is not hybridized, the reporter dye and the quencher remain in close proximity to each other, and due to Forster resonance energy transfer (FRET) fluorescence of the reporter dye is therefore quenched. In the PCR reaction, the polymerase extends the primers and replicates the template sequence to which the probe can hybridize. The hybridized probe is then cleaved by the 5′ exonuclease activity of a polymerase employed in the PCR reaction. This process releases the reporter dye away from close proximity to the quencher, resulting in an increase in fluorescence donor intensity. The fluorescent signals are then detected and employed for, for example, quantification of the target sequence.

However, probe cleavage by the 5′ exonuclease activity is limited by several factors. First, the probe must bind to the target before primer extension occludes the probe binding site. Second, the probe cleavage rate is primarily limited by the PCR efficiency, i.e., maximally cleavage of one probe molecule per round of amplification per target. Third, due to presence of primer dimers and/or loss of enzymatic activity of the polymerase during thermal cycling, the final plateau fluorescence intensity arising from samples having a low copy number is usually lower than from samples having a high copy number. This may lead to false negatives in the presence of low copy numbers of target sequences when the baseline threshold setting is not optimal.

Apart from 5′ exonuclease activity, probe can also be solely cleaved by endonucleases, including DNase, RNase, restriction endonuclease, etc. For example, U.S. Pat. Nos. 5,011,769 and 5,763,181, of which contents are incorporated herein by reference, describe a chimeric probe having a, DNA-RNA-DNA structure, labeled with either radioactive isotopes or a fluorescence reporter and quencher dye combination. Once hybridized to its single-stranded target sequence, the structure can be specifically recognized by RNase H endonuclease and the RNA portion of the probe cleaved. Degradation of the probe leads to an increase in donor fluorescence as the cleaved probe dissociates from the target at the reaction temperature and FRET is reversed. The kinetics of the change in donor signal intensity may be used to detect and/or quantify the target.

### SUMMARY

A method according to one or more embodiments of the invention incorporates both endonuclease and exonuclease activities to foster probe cleavage kinetics in a target sequence amplification process. It utilizes the 5′ exonuclease activity of a polymerase and endonuclease activity of, for example RNase H. The 5′ exonuclease activity and endonuclease activity both act on a probe which is a suitable substrate for both the 5′ exonuclease activity of a polymerase and endonuclease activity of RNase H. According to an embodiment, a suitable probe may be a dual labeled DNA-RNA-DNA probe. Presence of the RNase H enzyme may have little or no effect on PCR efficiency, but benefits in many aspects of the probe degradation process, particularly the PCR plateau signal intensity. As will be described hereinafter, the head-to-head comparison in the presence or absence of RNase H in real-time PCR assays revealed a significant improvement in signal-to-noise ratio. In particular, low fluorescence intensities that are usually associated with low concentrations of a target sequence in a technology using TaqMan® can be remediated and brought to the same plateau level as those of high concentrations of template. Therefore, the method according to embodiments of the invention can significantly eliminate false negatives due to low fluorescent signals.

The method according to one or more embodiments of the invention incorporates both endonuclease and exonuclease activities for probe cleavage as a means of detecting nucleic acids. Therefore, degradation of the probe is attributed to the 5′ exonuclease activity of Taq polymerase during primer extension, and endonuclease activity of RNase H. However, to be a suitable substrate for RNase H, the probe has a DNA-RNA-DNA chimeric structure. The addition of this endonuclease activity, in comparison to the exonuclease activity alone, increases the fluorescence signal-to-noise ratio and consequently improves detection sensitivity.

In an embodiment, there is provided a method of detecting a target sequence in a sample, the method including: (a) amplifying the target sequence in the sample to produce an increased number of copies of the target sequence, the amplification reaction including hybridizing a first primer and a second primer to the target nucleic acid in the sample to obtain a hybridized product of the target nucleic acid and the primers, and extending the first and the second primers of the hybridized product using a template-dependent nucleic acid polymerase to produce an extended primer product; (b) hybridizing the extended primer product to at least one probe oligonucleotide to obtain a hybridized product of the extended primer product: probe oligonucleotide, wherein the probe comprises a 5′-DNA sequence and an RNA sequence and coupled to a detectable label; (c) contacting the hybridized product of the extended primer product:probe oligonucleotide with both an RNase H and an exonuclease activity, said RNase and exonuclease activity being present simultaneously; and (d) detecting an increase in the emission of a signal from the label on the probe, wherein the increase in signal indicates the presence of the target sequence in the sample and wherein the intensity of the signal is higher by 1% or more compared to the signal intensity obtained from the same detection method performed in the absence of the RNase H. In embodiments, the signal intensity is greater by 10% or more, 20% or more, 30% or more, 40% or more, 50% or more, 60% or more, 70% or more, 80% or more, 85% or more, 90% or more, or 95% or more.

In an embodiment, the method comprises plural cycles of steps from (a) to (d) and each cycle comprises an exponential phase, a liner phase, and a plateau phase, wherein the signal intensity at the plateau phase is higher by 1% or more compared to the signal intensity obtained from the same method performed in the absence of the RNase H. In embodiments, the signal intensity is greater by 10% or more, 20% or more, 30% or more, 40% or more, 50% or more, 60% or more, 70% or more, 80% or more, 85% or more, 90% or more, or 95% or more.

The probe may be coupled to a fluorescence resonance energy transfer pair, one of the pair being coupled to the 3′ end of the probe and the other of the pair being coupled to the 5′ end of the probe.

In an embodiment, the amplification may be accomplished using a method selected from polymerase chain reaction. The amplification, hybridization, and the contacting are carried out simultaneously or sequentially.

In another embodiment, the method may further include culturing the sample containing the target sequence in an enriched medium before the amplification, to enhance growth of a pathogen containing the target sequence.

The target sequence may be a RNA. The method may further include a step of producing a complementary DNA sequence of the target RNA sequence, by for example reverse transcription.

In another embodiment, the target sequence may be a DNA.

In an embodiment, the exonuclease activity may be orginated from the DNA polymerase.

The RNase H may be RNase HII. Furthermore, the RNase H may be thermostable according to an embodiment.

When the probe has a 5′-DNA-RNA-DNA-3′ structure, the RNA sequence may have from 1 to 10 nucleotides. In an embodiment, the RNA sequence may have from 1 to 6 nucleotides. In another embodiment, the RNA sequence may have from 1 to 4 nucleotides.

### BRIEF DESCRIPTION OF DRAWINGS

The skilled artisan will understand that the drawings, described below, are for illustration purposes only. The figures are not intended to limit the scope of the teachings in any way.

FIG. 1 is a diagram showing the structure of a chimeric probe comprised of a DNA-RNA-DNA oligonucleotide probe to which a reporter dye and a quencher molecule are coupled to the each end of the oligonucleotide, respectively.

FIG. 2 illustrates several schemes of possible probe cleavage mechanisms in the presence of both endonuclease and exonuclease. A typical PCR reaction contains forward and reverse primers, polymerase, RNase H, probe and a target sequence. During PCR cycling, probe can be cleaved by the polymerase's 5′-3′ exonuclease activity during primer extension (scheme A), or endonuclease activity by RNase H (scheme B), or both polymerase and RNase H (scheme C). At the end of each PCR cycle, the nascent complementary strand is synthesized and, therefore, the probe binding site is occluded (scheme D).

FIG. 3 is a graph showing performance of PCR in the presence and absence of RNase H. A non-specific DNA intercalating dye, SYBR Green I, was used to monitor amplification of the target sequence. Inclusion of RNase H does not change the exponential amplification performance of the reaction.

FIG. 4 is a graph showing probe degradation kinetics, reflected by changes in fluorescence signals of the Cal Fluor Red 610 dye. A significant increase in the final plateau fluorescence intensity was observed when both Taq polymerase and RNase H were present, as compared to Taq polymerase alone.

FIG. 5 shows a comparison of TaqMan® (5A) and CataCleave™ (5B) detection of a dilution series of Salmonella invA plasmid in a real time PCR (“qPCR”) assay. At low target copy number (5 copies) the plateau fluorescence intensity was much higher in reactions containing RNase H. The final plateau fluorescence intensities of all the tested samples were similar in reactions including RNase H, as compared to TaqMan detection alone.

FIG. 6 is a graph comparing PCR efficiency by Taq polymerase, and by both Taq polymerase and RNase H.

FIG. 7 is a graph comparing probe cleavage by Taq polymerase, polymerase alone and by both Taq polymerase and RNase H. Probe cleavage due to hydrolysis was used as a background control.

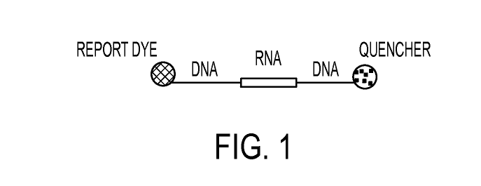

### DETAILED DESCRIPTION

The practice of the invention employs, unless otherwise indicated, conventional molecular biological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements; Sambrook, et al., Molecular Cloning: A Laboratory Manual, 2nd Edition, Cold Spring Harbor, N.Y. (1989).

Unless defined otherwise, all technical and scientific terms used herein have the same meaning as is commonly understood by one of skill in the art. The specification also provides definitions of terms to help interpret the disclosure and claims of this application. In the event a definition is not consistent with definitions elsewhere, the definition set forth in this application will control.

As used herein, the term “base” refers to any nitrogen-containing heterocyclic moiety capable of forming Watson-Crick type hydrogen bonds in pairing with a complementary base or base analog. A large number of natural and synthetic (non-natural, or unnatural) bases, base analogs and base derivatives are known. Examples of bases include purines, pyrimidines, and modified forms thereof. The naturally occurring bases include, but are not limited to, adenine (A), guanine (G), cytosine (C), uracil (U) and thymine (T). As used herein, it is not intended that the invention be limited to naturally occurring bases, as a large number of unnatural (non-naturally occurring) bases and their respective unnatural nucleotides that find use with the invention are known to one of skill in the art.

The term “nucleoside” refers to a compound consisting of a base linked to the C-1′ carbon of a sugar, for example, ribose or deoxyribose.

The term “nucleotide” refers to a phosphate ester of a nucleoside, as a monomer unit or within a polynucleotide. The term “nucleotide,” as used herein, refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR2 or halogen groups, where each R is independently H, C1-C6 alkyl or C5-C14 aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides, 2′-4′- and 3′-4′-linked and other “locked” or “LNA”, bicyclic sugar modifications (see, e.g., PCT published application nos. WO 98/22489, WO 98/39352, and WO 99/14226; and U.S. Pat. Nos. 6,268,490 and 6,794,499). Further synthetic nucleotides having modified base moieties and/or modified sugar moieties, e.g., as described by Scheit: Nucleotide Analogs (John Wiley New York, 1980); Uhlman and Peyman, 1990, Chemical Reviews 90:543-584.

The terms “polynucleotide,” “nucleic acid,” “oligonucleotide,” “oligomer,” “oligo,” primer or equivalent terms, as used herein refer to a polymeric arrangement of monomers that can be corresponded to a sequence of nucleotide bases, such as deoxyribonucleic acid (DNA), ribonucleic acid (RNA), and, where appropriate, phosphothioate containing nucleic acids, locked nucleic acid (LNA), peptide nucleic acid (PNA), or other derivative nucleic acid molecules and combinations thereof.

Nucleic acids include, but are not limited to, synthetic DNA, plasmid DNA, genomic DNA, cDNA, hnRNA, small nuclear snRNA, mRNA, rRNA, tRNA, miRNAs, fragmented nucleic acid, nucleic acid obtained from subcellular organelles such as mitochondria or chloroplasts, and nucleic acid obtained from microorganisms or DNA or RNA viruses that may be present on or in a biological sample. Nucleic acids may be composed of a single type of sugar moiety, e.g., as in the case of RNA and DNA, or mixtures of different sugar moieties, e.g., as in the case of RNA/DNA chimeras.

Polynucleotides are polymers of nucleotides comprising two or more nucleotides. Polynucleotides may be double-stranded nucleic acids, including annealed oligonucleotides wherein the second strand is an oligonucleotide with the reverse complement sequence of the first oligonucleotide, single-stranded nucleic acid polymers comprising deoxythymidine, single-stranded RNAs, double stranded RNAs or RNA/DNA heteroduplexes or single-stranded nucleic acid polymers comprising double stranded regions e.g. DNA hairpin loops and/or RNA hairpin loops and/or DNA/RNA hairpin loops.

As used herein, an “oligonucleotide” refers to a short polynucleotide. In certain embodiments, an oligonucleotide may be about 10, about 20, about 30, about 40, about 50 or more 60 nucleotides in length. In other embodiments, an oligonucleotide is less than about 500 nucleotides, less than about 250 nucleotides, less than about 200 nucleotides, less than about 150 nucleotide or less than 100 nucleotides.

Oligonucleotides or polynucleotides may be modified or may comprise modified bases or modified or non-naturally occurring sugar residues. Several reviews on modified oligonucleotides, including conjugates have been published; see for example, Verma and Eckstein Annu Rev. Biochem. (1998) 67:99-134, Uhlmann and Peyman, Chemical Reviews, Vol. 90, pgs. 543-584 (1990), and Goodchild, Bioconjugate Chemistry, Vol. 1, pgs 165-187 (1990), Cobb Org Biomol Chem. (2007) 5(20):3260-75, Lyer et al. Curr Opin Mol. Ther. (1999) 1(3):344-58), U.S. Pat. Nos. 6,172,208, 5,872,244 and published U.S. Patent Application No. 2007/0281308.

The term “template” or “template nucleic acid” refers to a plurality of nucleic acid molecules used as the starting material or template for amplification in a PCR reaction or reverse transcriptase-PCR reaction. Template nucleic acid sequences may include both naturally occurring and synthetic molecules. Exemplary template nucleic acid sequences include, but are not limited to, genomic DNA or genomic RNA.

A “target sequence,” “target DNA” or “target RNA” or “target nucleic acid,” or “target nucleic acid sequence” refers to a region of a template nucleic acid that is to be analyzed.

As used herein, the term “amplification primer” or “PCR primer” or “primer” refers to an enzymatically extendable oligonucleotide that comprises a defined sequence that is designed to hybridize in an antiparallel manner with a complementary, primer-specific portion of a target nucleic acid sequence. Thus, the primer, which is generally in molar excess relative to its target polynucleotide sequence, primes template-dependent enzymatic DNA synthesis and amplification of the target sequence. A primer nucleic acid does not need to have 100% complementarity with its template subsequence for primer elongation to occur; primers with less than 100% complementarity can be sufficient for hybridization and polymerase elongation to occur provided the penultimate base at the 3′ end of the primer is able to base pair with the template nucleic acid. A PCR primer is preferably, but not necessarily, synthetic, and will generally be approximately about 10 to about 100 nucleotides in length.

The term “probe” used herein refers to a nucleic acid having a sequence complementary to a target nucleic acid sequence and capable of hybridizing to the target nucleic acid to form a duplex. The sequence of the probe may be fully or completely complementary to the target nucleic acid sequence. The probe may be labeled so that the target nucleic acid may be detected simultaneously with PCR.

Oligonucleotides may be synthesized and prepared by any suitable method (such as chemical synthesis), which is known in the art. A number of computer programs (e.g., Primer-Express) are readily available to design optimal primer sets. One of the skilled artisans would therefore easily optimize and identify primers flanking a target nucleic acid sequence of interest. For example, synthesized primers can be between 20 and 26 base pairs in length with a melting point (T_{M}) of around 55 degrees. Commercially available primers may also be used to amplify a particular target nucleic acid sequence of interest. Hence, it will be apparent to one of skill in the art that the primers and probes based on the nucleic acid information provided (or publicly available with accession numbers) can be prepared accordingly.

The terms “annealing” and “hybridization” are used interchangeably and mean the base-pairing interaction of one nucleic acid with another nucleic acid that results in formation of a duplex, triplex, or other higher-ordered structure. In certain embodiments, the primary interaction is base-specific, e.g., A/T and G/C, by Watson/Crick and Hoogsteen-type hydrogen bonding. In certain embodiments, base-stacking and hydrophobic interactions may also contribute to duplex stability. “Substantially complimentary” refers to two nucleic acid strands that are sufficiently complimentary in sequence to anneal and form a stable duplex.

### Nucleic Acid Template Preparation

Nucleic acid templates can be derived from humans, non-human animals, plants, bacteria, fungi, protozoa, viruses and recombinant nucleic acids such as plasmid, phage or viral vectors.

In certain embodiments, the template nucleic acid is purified from a sample which may comprise prokaryotic or eukaryotic cells, cultured cells, human or animal fluid or tissues including, but not limited to, blood, saliva, sputum, urine, feces, skin cells, hair follicles, semen, vaginal fluid, bone fragments, bone marrow, brain matter, cerebrospinal fluid, amniotic fluid, and the like. Samples may also include bacterial cells or spores (including gram+ or gram−), and viruses (including DNA-based and RNA-based). In some embodiments, the samples may be collected using swab sampling of surfaces.

Procedures for the extraction and purification of nucleic acids from samples are well known in the art (as described in Sambrook J et. al. Molecular Cloning, Cold Spring harbor Laboratory Press (1989), Ausubel et al. Short Protocols in Molecular Biology, 5th Ed. (2002) John Wiley & Sons, Inc. New York).

In addition, several commercial kits are available for the isolation of nucleic acids. Exemplary kits include, but are not limited to, Puregene DNA isolation kit (PG) (Gentra Systems, Inc., Minneapolis, Minn.), Generation Capture Column kit (GCC) (Gentra Systems, Inc.), MasterPure DNA purification kit (MP) (Epicentre Technologies, Madison, Wis.), Isoquick nucleic acid extraction kit (IQ) (Epoch Pharmaceuticals, Bothell, Wash.), NucliSens isolation kit (NS) (Organon Teknika Corp., Durham, N.C.), QIAamp DNA Blood Mini Kit (Qiagen; Cat. No. 51104), MagNA Pure Compact Nucleic Acid Isolation Kit (Roche Applied Sciences; Cat. No. 03730964001), Stabilized Blood-to-CTTM Nucleic Acid Preparation Kit for qPCR (Invitrogen, Cat. No. 4449080) and GF-1 Viral Nucleic Acid Extraction Kit (GeneOn, Cat. No. RD05).

### The Nucleic Acid Polymerase

The nucleic acid polymerase can have one or more of the activities of a DNA-dependent DNA polymerase, a DNA-dependent RNA polymerase, a RNA-dependent DNA polymerase or a RNA dependent RNA polymerase.

A “DNA-dependent DNA polymerase activity” refers to the activity of a DNA polymerase enzyme that uses deoxyribonucleic acid (DNA) as a template for the synthesis of a complementary and anti-parallel DNA strand.

A “DNA-dependent RNA polymerase activity” refers to the activity of an RNA polymerase enzyme that uses deoxyribonucleic acid (DNA) as a template for the synthesis of an RNA strand in a process called “transcription.” (for example, Thermo T7 RNA polymerase, commercially available from Toyobo Life Science Department, Catalogue No. TRL-201)

A “RNA-dependent DNA polymerase activity” refers to the activity of a DNA polymerase enzyme that uses ribonucleic acid (RNA) as a template for the synthesis of a complementary and anti-parallel DNA strand in a process called “reverse transcription.” (see below)

A “RNA-dependent RNA polymerase activity” refers to the activity of a RNA polymerase enzyme that uses ribonucleic acid (RNA) as a template for the synthesis of a complementary RNA strand (for example, Thermus thermophilus RNA polymerase, commercially available from Cambio, Catalogue No. T90250).

In certain embodiments, the nucleic acid polymerase is a thermostable polymerase that may have more than one of the above-specified catalytic activities.

As used herein, the term “thermostable,” as applied to an enzyme, refers to an enzyme that retains its biological activity at elevated temperatures (e.g., at 55° C. or higher), or retains its biological activity following repeated cycles of heating and cooling.

As used herein, an “amplifying polymerase activity” refers to an enzymatic activity that catalyzes the polymerization of deoxyribonucleotides or ribonucleotides. Generally, the enzyme will initiate synthesis at the 3′-end of the primer annealed to a target nucleic acid template sequence, and will proceed toward the 5′ end of the template strand.

Non-limiting examples of thermostable DNA polymerases may include, but are not limited to, polymerases isolated from the thermophilic bacteria Thermus aquaticus (Taq polymerase), Thermus thermophilus (Tth polymerase), Thermococcus litoralis (Tli or VENT™ polymerase), Pyrococcus furiosus (Pfu or DEEPVENT™ polymerase), Pyrococcus woosii (Pwo polymerase) and other Pyrococcus species, Bacillus stearothermophilus (Bst polymerase), Sulfolobus acidocaldarius (Sac polymerase), Thermoplasma acidophilum (Tac polymerase), Thermus rubber (Tru polymerase), Thermus brockianus (DYNAZYME™ polymerase) i (Tne polymerase), Thermotoga maritime (Tma) and other species of the Thermotoga genus (Tsp polymerase), and Methanobacterium thermoautotrophicum (Mth polymerase). The PCR reaction may contain more than one thermostable polymerase enzyme with complementary properties leading to more efficient amplification of target sequences. For example, a nucleotide polymerase with high processivity (the ability to copy large nucleotide segments) may be complemented with another nucleotide polymerase with proofreading capabilities (the ability to correct mistakes during elongation of target nucleic acid sequence), thus creating a PCR reaction that can copy a long target sequence with high fidelity. The thermostable polymerase may be used in its wild type form. Alternatively, the polymerase may be modified to contain a fragment of the enzyme or to contain a mutation that provides beneficial properties to facilitate the PCR reaction.

In one embodiment, the thermostable polymerase may be Taq DNA polymerase. Many variants of Taq polymerase with enhanced properties are known and include, but are not limited to, AmpliTaq™, AmpliTaq™, Stoffel fragment, SuperTaq™, SuperTaq™ plus, LA Taq™, LApro Taq™, and EX Taq™. In another embodiment, the thermostable polymerase is the AmpliTaq Stoffel fragment.

### The “Substantially Double-Stranded Oligonucleotide”

The “substantially double-stranded oligonucleotide” refers to an oligonucleotide having at least one region that has a strand with one or more nucleotides engaged in complementary hydrogen bond pairs with one or more nucleotides of a region of another strand. Base-pairing in the substantially double-stranded region may comprise one or more contiguously base paired nucleotides or contiguously base paired nucleotides interspersed with one or more nucleotides that are not base-paired. A substantially double-stranded region may comprise one or more ribonucleotides or deoxyribonucleotides. The based-paired nucleotides may be situated at the 5′ end or 3′ end of one of the strands of the “substantially double-stranded oligonucleotide” or anywhere in between. The base-pairing in the substantially double-stranded region may be intermolecular or intermolecular. That is, the base pairing may be between two or more separate oligonucleotides (e.g., double-stranded oligonucleotides), or within a single oligonucleotide to form a stem and loop structure (e.g., hairpin oligonucleotides). In one embodiment the “substantially double-stranded oligonucleotide” may comprise 1, 2, 3, 4, 5 or more stem structures. The “substantially double-stranded oligonucleotide” may comprise about 1, about 2, about 3, about 4, about 5, about 6, about 7, about 8, about 9, about 10, about 15, about 20, about 25, about 50 or more base paired nucleotides. The “substantially double-stranded oligonucleotide” may comprise about 1, about 2, about 3, about 4, about 5, about 6, about 7, about 8, about 9, about 10, about 15, about 20, about 25, about 50 or more nucleotides that are not base paired.

In some embodiments, the “substantially double-stranded oligonucleotide” comprises two based paired oligonucleotides that are 100% complimentary to each other. In other embodiments, the “substantially double-stranded oligonucleotide” comprises two based paired oligonucleotides may have about 95%, about 90%, about 75%, about 50%, or less complementarity to each other.

The 5′ end or 3′ end of one of the strands in the “substantially double-stranded oligonucleotide” may have a 5′ or 3′ overhang at one or both ends of the oligonucleotide or one or both ends of the oligonucleotide may be base-paired and blunt-ended.

The amount of double-stranded oligonucleotide present can be estimated by knowing the oligonucleotide's melting temperature or Tm, i.e. the temperature at which about 50% of the oligonucleotide and its complement are in duplex.

The melting temperature (Tm) of a double stranded region of an oligonucleotide can be calculated from the oligonucleotide sequence using methods that are well known in the art.

For example, the Tm of an oligonucleotide can be calculated using the formulas, for example:

Tₘ=4° C.×(number of G's and C's in the primer)+2° C.×(number of A's and T's in the primer)

This formula is valid for oligonucleotides having a double stranded region of <14 bases and assumes that the reaction is carried out in the presence of 50 mM monovalent cations.

For longer oligonucleotides having a double stranded region >14 bases, the formula below can be used:

Tₘ=64.9° C.+41° C.×(number of G's and C's in the primer−16.4)/N

Where N is the length of the primer.

Another commonly used formula takes into account the salt concentration of the reaction (see Rychlik, W. and Rhoads, R. E. (1989) Nucl. Acids Res. 17, 8543; PCR Core Systems Technical Bulletin #TB254, Promega Corporation; Sambrook, J., Fritsch, E. F. and Maniatis, T. (1989) Molecular Cloning: A Laboratory Manual, Cold Spring Harbor Laboratory Press, Cold Spring Harbor, N.Y. and Mueller, P. R. et al. (1993) In: Current Protocols in Molecular Biology 15.5, Greene Publishing Associates, Inc. and John Wiley and Sons, New York).

This formula is as follows:

Tₘ=81.5° C.+16.6° C.×(log₁₀[Na⁺]+[K⁺])+0.41° C.×(% GC)−675/N

Where N is the number of nucleotides in the oligonucleotide.

The most sophisticated Tₘ calculations take into account the exact sequence and base stacking parameters, not just the base composition (as described in Borer P. N. et al. (1974) J. Mol. Biol. 86, 843; SantaLucia, J. (1998) Proc. Nat. Acad. Sci. USA 95, 1460; Allawi, H. T. and SantaLucia, J. Jr. (1997) Biochemistry 36, 10581 and von Ahsen N. et al. (1999) Clin. Chem. 45, 2094).

The equation used is:

$${T}_{m} = \frac{Δ H \frac{kcal}{° C . * Mol}}{Δ S + R ln ( [ primer ] / 2 )} - 275.15 ° C .$$

Where, ΔH is the enthalpy of base stacking interactions adjusted for helix initiation factors, ΔS is the entropy of base stacking adjusted for helix initiation factors and for the contributions of salts to the entropy of the system and R is the universal gas constant (1.987 Cal/° C. mole).

This equation, as implemented above, is valid if the following assumptions are met:
The primer is not self complementary. For self-complementary oligonucleotides, the denominator of the equation becomes ΔS+R ln([primer]/4)
The primer concentration is much greater than the target concentration.

If the concentrations are almost equal, the denominator of the equation becomes ΔS+R ln([primer]−[target]/2)
The primer is an “oligonucleotide” rather than a long polymer. The salt effects on polymers is significantly different from those on oligos. For a complete discussion, see reference 6.

The melting temperature for different oligonucleotides can be conveniently determined using web-based calculators freely available on the web, e.g. the Biomath calculator on Promega's web site

In one embodiment, the “substantially double-stranded oligonucleotide” has a Tm from about 50° C. to about 55° C. or about 60° C.

The appropriate Tm is selected to ensure the oligonucleotide is double-stranded at a temperature
(1) where it is desirable to inhibit the nucleic polymerase's catalytic activity, i.e. at temperatures from about 20° C. to about 50° C., and
(2) where the heat-activated hot start nuclease can cleave and disrupt the secondary structure of the substantially double-stranded oligonucleotide sufficiently to preclude the inhibition of the nucleic polymerase's catalytic activity.

In some embodiments, the “substantially double-stranded oligonucleotide” is chemically modified at its 3′-end and/or 5′-end. The modification of the oligonucleotide at its 3′ end blocks the oligonucleotide from participating in primer extension.

In one embodiment, the oligonucleotide is blocked from participating in primer extension through the incorporation of a dideoxyribonucleotide at the 3′ end.

For example, the 3′-terminus of a double stranded oligonucleotide may be capped at the 3′ terminus with a dideoxythymine triphosphate using a Klenow fragment mutant (F762Y) of DNA polymerase I (Escherichia coli) or T7 DNA polymerase (Tabor, S, and Richardson, C. C., 1995, Proc. Natl. Acad. Sci. USA 92, 6339-6343). The 3′-OH terminus of the oligonucleotide is then extended with ddTTP by the polymerase at 20 μM ddTTP in the presence of 2 mM Mg2+ in 50 mM Tris pH 7.5 buffer, at 37° C. for 30 min. Following extension, the sample is placed in a 100° C. water bath for 3 min to denature the protein. Following heating the oligonucleotide sample was cooled slowly to ambient temperature (2-3 hrs) to allow formation of duplex structure.

In another embodiment, the substantially double-stranded oligonucleotide contains at least one RNA:DNA base pair that can be endonucleolytically cleaved by an RNase H activity. The location of the at least one RNA:DNA base pair is not particularly important provided any double stranded fragments produced by the cleavage of the double stranded oligonucleotide at the at least one RNA:DNA base pair have a Tm that is at least about 5-10° C. below the temperature of the PCR reaction's annealing step. Hence, any fragments of the double-stranded oligonucleotide produced by the cleavage are unable to interfere with the subsequent PCR reaction because, at temperatures above 55° C., the fragments remain single-stranded and are unable to either inhibit the nucleic acid polymerase or anneal to a DNA template and provide a substrate for primer extension.

In certain embodiments, the oligonucleotide may have one or more blocking agents. A blocking agent refers to a nucleotide (or derivatives thereof), modified oligonucleotides and/or one or more other modifications which are incorporated into the nucleic acid inhibitors of the invention to prevent or inhibit degradation or digestion of such nucleic acid molecules by DNase activity.

### Hot Start Nuclease

As used herein, a “hot start” enzyme composition refers to compositions having an enzymatic activity that is inhibited at non-permissive temperatures, i.e. from about 25° C. to about 45° C. and activated or ‘heat inducible’ at temperatures compatible with a PCR reaction, e.g. from about 55° C. to about 95° C.

A “hot start” nuclease composition, as used herein, refers to a composition comprising an ‘inducible’ RNA or DNA endonuclease, e.g. heat-inducible, that when activated, can cleave specifically the double-stranded oligonucleotide.

In a preferred embodiment, the “hot start” nuclease is a ‘hot start’ RNase H that can cleave a double-stranded oligonucleotide comprising at least one RNA:DNA base pair.

In some embodiments, the Tm of an oligonucleotide is determined using the SYBR Green intercalating dye. At low temperature the oligonucleotide inhibitors described herein fold upon themselves to form a double stranded hairpin structure. SYBR Green when intercalated into this double stranded structure absorbs light at 497 nM and emits at 520 nM. 520 nM light emission is monitored as the sample is slowly heated. Near the melting point (Tm) the oligonucleotide slowly unfolds releasing free SYBR Green causing a net loss in total fluorescence. The Tm is the point at which 50% of the oligonucleotide is in its melted state.

RNase H hydrolyzes RNA in RNA-DNA hybrids. First identified in calf thymus, RNase H has subsequently been described in a variety of organisms. RNase H activity appears to be ubiquitous in eukaryotes and bacteria. Although RNase Hs form a family of proteins of varying molecular weight and nucleolytic activity, substrate requirements appear to be similar for the various isotypes. For example, most RNase Hs studied to date function as endonucleases and require divalent cations (e.g., Mg²⁺, Mn²⁺) to produce cleavage products with 5′ phosphate and 3′ hydroxyl termini.

In prokaryotes, RNase H have been cloned and extensively characterized (see Crooke, et al., (1995) Biochem J, 312 (Pt 2), 599-608; Lima, et al., (1997) J Biol Chem, 272, 27513-27516; Lima, et al., (1997) Biochemistry, 36, 390-398; Lima, et al., (1997) J Biol Chem, 272, 18191-18199; Lima, et al., (2007) Mol Pharmacol, 71, 83-91; Lima, et al., (2007) Mol Pharmacol, 71, 73-82; Lima, et al., (2003) J Biol Chem, 278, 14906-14912; Lima, et al., (2003) J Biol Chem, 278, 49860-49867; Itaya, M., Proc. Natl. Acad. Sci. USA, 1990, 87, 8587-8591). For example, E. coli RNase HII is 213 amino acids in length whereas RNase HI is 155 amino acids long. E. coli RNase HII displays only 17% homology with E. coli RNase HI. An RNase H cloned from S. typhimurium differed from E. coli RNase HI in only 11 positions and was 155 amino acids in length (Itaya, M. and Kondo K., Nucleic Acids Res., 1991, 19, 4443-4449).

Proteins that display RNase H activity have also been cloned and purified from a number of viruses, other bacteria and yeast (Wintersberger, U. Pharmac. Ther., 1990, 48, 259-280). In many cases, proteins with RNase H activity appear to be fusion proteins in which RNase H is fused to the amino or carboxy end of another enzyme, often a DNA or RNA polymerase. The RNase H domain has been consistently found to be highly homologous to E. coli RNase HI, but because the other domains vary substantially, the molecular weights and other characteristics of the fusion proteins vary widely.

In higher eukaryotes two classes of RNase H have been defined based on differences in molecular weight, effects of divalent cations, sensitivity to sulfhydryl agents and immunological cross-reactivity (Busen et al., Eur. J. Biochem., 1977, 74, 203-208). RNase HI enzymes are reported to have molecular weights in the 68-90 kDa range, be activated by either Mn²⁺ or Mg²⁺ and be insensitive to sulfhydryl agents. In contrast, RNase HII enzymes have been reported to have molecular weights ranging from 31-45 kDa, to require Mg²⁺ to be highly sensitive to sulfhydryl agents and to be inhibited by Mn²⁺ (Busen, W., and Hausen, P., Eur. J. Biochem., 1975, 52, 179-190; Kane, C. M., Biochemistry, 1988, 27, 3187-3196; Busen, W., J. Biol. Chem., 1982, 257, 7106-7108).

An enzyme with RNase HII characteristics has also been purified to near homogeneity from human placenta (Frank et al., Nucleic Acids Res., 1994, 22, 5247-5254). This protein has a molecular weight of approximately 33 kDa and is active in a pH range of 6.5-10, with a pH optimum of 8.5-9. The enzyme requires Mg²⁺ and is inhibited by Mn²⁺ and n-ethyl maleimide. The products of cleavage reactions have 3′ hydroxyl and 5′ phosphate termini.

A detailed comparison of RNases from different species is reported in Ohtani N, Haruki M, Morikawa M, Kanaya S. J Biosci Bioeng. 1999; 88(1):12-9.

Examples of RNase H enzymes, which may be employed in the embodiments, also include, but are not limited to, thermostable RNase H enzymes isolated from thermophilic organisms such as Pyrococcus furiosus, Pyrococcus horikoshi, Thermococcus litoralis or Thermus thermophilus.

Other RNase H enzymes that may be employed in the embodiments are described in, for example, U.S. Pat. No. 7,422,888 to Uemori or the published U.S. Patent Application No. 2009/0325169 to Walder, the contents of which are incorporated herein by reference.

In one embodiment, thermostable RNase H enzymes disclosed in commonly owned application Ser. No. 13/108,311, of which content is incorporated herein by reference, may be used.

The homology can be determined using, for example, a computer program DNASIS-Mac (Takara Shuzo), a computer algorithm FASTA (version 3.0; Pearson, W. R. et al., Pro. Natl. Acad. Sci., 85:2444-2448, 1988) or a computer algorithm BLAST (version 2.0, Altschul et al., Nucleic Acids Res. 25:3389-3402, 1997).

### RNase H

In certain embodiments, the RNase H can be modified to produce a ‘hot start’ RNase H.

The term “modified RNase H,” as used herein, can be an RNase H reversely coupled to or reversely bound to an inhibiting factor that causes the loss of the endonuclease activity of the RNase H. Release or decoupling of the inhibiting factor from the RNase H restores at least partial or full activity of the endonuclease activity of the RNase H. About 30-100% of its activity of an intact RNase H may be sufficient. The inhibiting factor may be a ligand or a chemical modification. The ligand can be an antibody, an aptamer, a receptor, a cofactor, or a chelating agent. The ligand can bind to the active site of the RNase H enzyme thereby inhibiting enzymatic activity or it can bind to a site remote from the RNase's active site. In some embodiments, the ligand may induce a conformational change. The chemical modification can be a crosslinking (for example, by formaldehyde) or acylation. The release or decoupling of the inhibiting factor from the RNase H may be accomplished by heating a sample or a mixture containing the coupled RNase H (inactive) to a temperature of about 65° C. to about 95° C. or higher, and/or lowering the pH of the mixture or sample to about 7.0 or lower.

As used herein, a ‘hot start’ RNase H activity refers to the herein described modified RNase H that has an endonuclease catalytic activity that can be regulated by association with a ligand. Under permissive conditions, the RNase H endonuclease catalytic activity is activated whereas at non-permissive conditions, this catalytic activity is inhibited. In some embodiments, the catalytic activity of a modified RNase H can be inhibited at temperature conducive for reverse transcription, i.e. about 42° C., and activated at more elevated temperatures found in PCR reactions, i.e. about 65° C. to 95° C. A modified RNase H with these characteristics is said to be “heat inducible.”

In other embodiments, the catalytic activity of a modified RNase H can be regulated by changing the pH of a solution containing the enzyme.

### Modifications of RNase H

Crosslinking of RNase H enzymes can be performed using, for example, formaldehyde. In one embodiment, a thermostable RNase H is subjected to controlled and limited crosslinking using formaldehyde. By heating an amplification reaction composition, which comprises the modified RNase H in an active state, to a temperature of about 95° C. or higher for an extended time, for example about 15 minutes, the crosslinking is reversed and the RNase H activity is restored.

In general, the lower the degree of crosslinking, the higher the endonuclease activity of the enzyme is after reversal of crosslinking. The degree of crosslinking may be controlled by varying the concentration of formaldehyde and the duration of crosslinking reaction. For example, about 0.2% (w/v), about 0.4% (w/v), about 0.6% (w/v), or about 0.8% (w/v) of formaldehyde may be used to crosslink an RNase H enzyme. About 10 minutes of crosslinking reaction using 0.6% formaldehyde may be sufficient to inactivate RNase HII from Pyrococcus furiosus.

The crosslinked RNase H does not show any measurable endonuclease activity at about 37° C. In some cases, a measurable partial reactivation of the crosslinked RNase H may occur at a temperature of around 50° C., which is lower than the PCR denaturation temperature. To avoid such unintended reactivation of the enzyme, it may be required to store or keep the modified RNase H at a temperature lower than 50° C. until its reactivation.

In general, PCR requires heating the amplification composition at each cycle to about 95° C. to denature the double stranded target sequence which will also release the inactivating factor from the RNase H, partially or fully restoring the activity of the enzyme.

RNase H may also be modified by subjecting the enzyme to acylation of lysine residues using an acylating agent, for example, a dicarboxylic acid. Acylation of RNase H may be performed by adding cis-aconitic anhydride to a solution of RNase H in an acylation buffer and incubating the resulting mixture at about 1-20° C. for 5-30 hours. In one embodiment, the acylation may be conducted at around 3-8° C. for 18-24 hours. The type of the acylation buffer is not particularly limited. In an embodiment, the acylation buffer has a pH of between about 7.5 to about 9.0.

The activity of acylated RNase H can be restored by lowering the pH of the amplification composition to about 7.0 or less. For example, when Tris buffer is used as a buffering agent, the composition may be heated to about 95° C., resulting in the lowering of pH from about 8.7 (at 25° C.) to about 6.5 (at 95° C.).

The duration of the heating step in the amplification reaction composition may vary depending on the modified RNase H, the buffer used in the PCR, and the like. However, in general, heating the amplification composition to 95° C. for about 30 seconds-4 minutes is sufficient to restore RNase H activity. In one embodiment, using a commercially available buffer and one or more non-ionic detergents, full activity of Pyrococcus furiosus RNase HII is restored after about 2 minutes of heating.

RNase H activity may be determined using methods that are well in the art. For example, according to a first method, the unit activity is defined in terms of the acid-solubilization of a certain number of moles of radiolabeled polyadenylic acid in the presence of equimolar polythymidylic acid under defined assay conditions (see Epicentre Hybridase thermostable RNase HI). In the second method, unit activity is defined in terms of a specific increase in the relative fluorescence intensity of a reaction containing equimolar amounts of the probe and a complementary template DNA under defined assay conditions.

### Other ‘Hot Start’ Nucleases

In other embodiments, the hot start nuclease may be a heat-inducible thermostable sequence-specific endonuclease, such as a Type II Restriction Enzyme, that can cleave the ‘substantially double-stranded oligonucleotide” but not sequences found within the targeted nucleic sequence to be amplified.

A number of thermostable restriction enzymes have been isolated from extremophile microorganisms. For example, the thermostable restriction endonuclease, PspGI, was purified from Pyrococcus sp. strain GI-H. PspGI is an isoschizomer of EcoR11 and cleaves DNA before the first C in the sequence 5′ CCWGG 3′ (W is A or T). PspGI digestion can be carried out at 65 to 85° C. Recombinant PspGI has a half-life of 2 h at 95° C.

In certain embodiments, a thermostable restriction enzyme can be rendered ‘heat inducible’ by reversely coupling it to or reversely binding it to an inhibiting factor that causes the loss of the enzyme's catalytic activity at non-permissive temperatures. The inhibiting factor may be a ligand or a chemical modification. The ligand can be an antibody, an aptamer, a receptor, a cofactor, or a chelating agent. The ligand can bind to the active site of the thermostable restriction enzyme thereby inhibiting enzymatic activity or it can inhibit the catalytic activity by binding to a site remote from the thermostable restriction enzyme's active site. In some embodiments, the ligand may induce a conformational change. In another embodiment, the restriction enzyme may be modified by crosslinking (for example, by formaldehyde) or acylation. The release or decoupling of the inhibiting factor from the thermostable restriction enzyme may be accomplished by heating a sample or a mixture containing the coupled thermostable restriction enzyme (inactive) to a temperature of about 65° C. to about 95° C. or higher.

### PCR Amplification of Target Nucleic Acid Sequences

Nucleic acid amplification can be accomplished by a variety of methods, including, but not limited to, the polymerase chain reaction (PCR), nucleic acid sequence based amplification (NASBA), ligase chain reaction (LCR), strand displacement amplification (SDA) reaction, transcription mediated amplification (TMA) reaction, and rolling circle amplification (RCA). The polymerase chain reaction (PCR) is the method most commonly used to amplify specific target DNA sequences.

“Polymerase chain reaction,” or “PCR,” generally refers to a method for amplification of a desired nucleotide sequence in vitro. Generally, the PCR process consists of introducing a molar excess of two or more extendable oligonucleotide primers to a reaction mixture comprising a sample having the desired target sequence(s), where the primers are complementary to opposite strands of the double stranded target sequence. The reaction mixture is subjected to a program of thermal cycling in the presence of a DNA polymerase, resulting in the amplification of the desired target sequence flanked by the DNA primers.

PCR amplification may have three phases: exponential phase, linear phase and plateau phase. The exponential phase is the first phase of PCR amplification. During this exponential phase, reaction components are in excess. Assuming 100% reaction efficiency, there is an exact doubling of product each cycle, and the reaction is specific and precise. The linear phase is the second phase of PCR amplification, during which the reaction components are continuously being consumed but become limiting, amplification therefore slows and the reactions become highly variable, The final phase of PCR amplification is the plateau phase. At the plateau phase, the reaction components are insufficient for amplification and very few or no products are being generated.

The technique of PCR is described in numerous publications, including, PCR: A Practical Approach, M. J. McPherson, et al., IRL Press (1991), PCR Protocols: A Guide to Methods and Applications, by Innis, et al., Academic Press (1990), and PCR Technology: Principals and Applications for DNA Amplification, H. A. Erlich, Stockton Press (1989). PCR is also described in many U.S. patents, including U.S. Pat. Nos. 4,683,195; 4,683,202; 4,800,159; 4,965,188; 4,889,818; 5,075,216; 5,079,352; 5,104,792; 5,023,171; 5,091,310; and 5,066,584, each of which is herein incorporated by reference.

The term “sample” refers to any substance containing nucleic acid material.

As used herein, the term “PCR fragment” or “reverse transcriptase-PCR fragment” or “amplicon” refers to a polynucleotide molecule (or collectively the plurality of molecules) produced following the amplification of a particular target nucleic acid. A PCR fragment is typically, but not exclusively, a DNAPCR fragment. A PCR fragment can be single-stranded or double-stranded, or in a mixture thereof in any concentration ratio. A PCR fragment or RT-PCT can be about 100 to about 500 nt or more in length.

A “buffer” is a compound added to an amplification reaction which modifies the stability, activity, and/or longevity of one or more components of the amplification reaction by regulating the pH of the amplification reaction. The buffering agents of the invention are compatible with PCR amplification and site-specific RNase H cleavage activity. Certain buffering agents are well known in the art and include, but are not limited to, Tris, Tricine, MOPS (3-(N-morpholino) propanesulfonic acid), and HEPES (4-(2-hydroxyethyl)-1-piperazineethanesulfonic acid). In addition, PCR buffers may generally contain up to about 70 mM KCl and about 1.5 mM or higher MgCl₂, to about 50-200 μM each of nucleotides dATP, dCTP, dGTP and dTTP. The buffers of the invention may contain additivies to optimize efficient reverse transcriptase-PCR or PCR reaction.

An additive is a compound added to a composition which modifies the stability, activity, and/or longevity of one or more components of the composition. In certain embodiments, the composition is an amplification reaction composition. In certain embodiments, an additive inactivates contaminant enzymes, stabilizes protein folding, and/or decreases aggregation. Exemplary additives that may be included in an amplification reaction include, but are not limited to, betaine, formamide, KCl, CaCl₂, MgOAc, MgCl₂, NaCl, NH₄OAc, NaI, Na(CO₃)₂, LiCl, MnOAc, NMP, trehalose, demethylsulfoxide (“DMSO”), glycerol, ethylene glycol, dithiothreitol (“DTT”), pyrophosphatase (including, but not limited to Thermoplasma acidophilum inorganic pyrophosphatase (“TAP”)), bovine serum albumin (“BSA”), propylene glycol, glycinamide, CHES, Percoll™, aurintricarboxylic acid, Tween 20, Tween 21, Tween 40, Tween 60, Tween 85, Brij 30, NP-40, Triton X-100, CHAPS, CHAPSO, Mackernium, LDAO (N-dodecyl-N,N-dimethylamine-N-oxide), Zwittergent 3-10, Xwittergent 3-14, Xwittergent SB 3-16, Empigen, NDSB-20, T4G32, E. Coli SSB, RecA, nicking endonucleases, 7-deazaG, dUTP, UNG, anionic detergents, cationic detergents, non-ionic detergents, zwittergent, sterol, osmolytes, cations, and any other chemical, protein, or cofactor that may alter the efficiency of amplification. In certain embodiments, two or more additives are included in an amplification reaction. According to the invention, additives may be added to improve selectivity of primer annealing provided the additives do not interfere with the activity of RNase H.

### Reverse Transcriptase-PCR Amplification of a RNA Target Nucleic Acid Sequence

One of the most widely used techniques to study gene expression exploits first-strand cDNA for mRNA sequence(s) as template for amplification by the PCR.

The term “reverse transcriptase activity” and “reverse transcription” refers to the enzymatic activity of a class of polymerases characterized as RNA-dependent DNA polymerases that can synthesize a DNA strand (i.e., complementary DNA, cDNA) utilizing an RNA strand as a template.

“Reverse transcriptase-PCR” of “RNA PCR” is a PCR reaction that uses RNA template and a reverse transcriptase, or an enzyme having reverse transcriptase activity, to first generate a single stranded DNA molecule prior to the multiple cycles of DNA-dependent DNA polymerase primer elongation. Multiplex PCR refers to PCR reactions that produce more than one amplified product in a single reaction, typically by the inclusion of more than two primers in a single reaction.

Exemplary reverse transcriptases include, but are not limited to, the Moloney murine leukemia virus (M-MLV) RT as described in U.S. Pat. No. 4,943,531, a mutant form of M-MLV-RT lacking RNase H activity as described in U.S. Pat. No. 5,405,776, bovine leukemia virus (BLV) RT, Rous sarcoma virus (RSV) RT, Avian Myeloblastosis Virus (AMV) RT and reverse transcriptases disclosed in U.S. Pat. No. 7,883,871.

The reverse transcriptase-PCR procedure, carried out as either an end-point or real-time assay, involves two separate molecular syntheses: (i) the synthesis of cDNA from an RNA template; and (ii) the replication of the newly synthesized cDNA through PCR amplification. To attempt to address the technical problems often associated with reverse transcriptase-PCR, a number of protocols have been developed taking into account the three basic steps of the procedure: (a) the denaturation of RNA and the hybridization of reverse primer; (b) the synthesis of cDNA; and (c) PCR amplification. In the so called “uncoupled” reverse transcriptase-PCR procedure (e.g., two step reverse transcriptase-PCR), reverse transcription is performed as an independent step using the optimal buffer condition for reverse transcriptase activity. Following cDNA synthesis, the reaction is diluted to decrease MgCl₂, and deoxyribonucleoside triphosphate (dNTP) concentrations to conditions optimal for Taq DNA Polymerase activity, and PCR is carried out according to standard conditions (see U.S. Pat. Nos. 4,683,195 and 4,683,202). By contrast, “coupled” RT PCR methods use a common buffer optimized for reverse transcriptase and Taq DNA Polymerase activities. In one version, the annealing of reverse primer is a separate step preceding the addition of enzymes, which are then added to the single reaction vessel. In another version, the reverse transcriptase activity is a component of the thermostable Tth DNA polymerase Annealing and cDNA synthesis are performed in the presence of Mn²⁺ then PCR is carried out in the presence of Mg²⁺ after the removal of Mn²⁺ by a chelating agent. Finally, the “continuous” method (e.g., one step reverse transcriptase-PCR) integrates the three reverse transcriptase-PCR steps into a single continuous reaction that avoids the opening of the reaction tube for component or enzyme addition. Continuous reverse transcriptase-PCR has been described as a single enzyme system using the reverse transcriptase activity of thermostable Taq DNA Polymerase and Tth polymerase and as a two enzyme system using AMV RT and Taq DNA Polymerase wherein the initial 65° C. RNA denaturation step may be omitted.

In certain embodiments, one or more primers may be labeled. As used herein, “label,” “detectable label,” or “marker,” or “detectable marker”, which are interchangeably used in the specification, refers to any chemical moiety attached to a nucleotide, nucleotide polymer, or nucleic acid binding factor, wherein the attachment may be covalent or non-covalent. Preferably, the label is detectable and renders the nucleotide or nucleotide polymer detectable to the practitioner of the invention. Detectable labels include luminescent molecules, chemiluminescent molecules, fluorochromes, fluorescent quenching agents, colored molecules, radioisotopes or scintillants. Detectable labels also include any useful linker molecule (such as biotin, avidin, streptavidin, HRP, protein A, protein G, antibodies or fragments thereof, Grb2, polyhistidine, Ni²⁺, FLAG tags, myc tags), heavy metals, enzymes (examples include alkaline phosphatase, peroxidase and luciferase), electron donors/acceptors, acridinium esters, dyes and calorimetric substrates. It is also envisioned that a change in mass may be considered a detectable label, as is the case of surface plasmon resonance detection. The skilled artisan would readily recognize useful detectable labels that are not mentioned above, which may be employed in the operation of the present invention.

One step reverse transcriptase-PCR provides several advantages over uncoupled reverse transcriptase-PCR. One step reverse transcriptase-PCR requires less handling of the reaction mixture reagents and nucleic acid products than uncoupled reverse transcriptase-PCR (e.g., opening of the reaction tube for component or enzyme addition in between the two reaction steps), and is therefore less labor intensive, reducing the required number of person hours. One step reverse transcriptase-PCR also requires less sample, and reduces the risk of contamination. The sensitivity and specificity of one-step reverse transcriptase-PCR has proven well suited for studying expression levels of one to several genes in a given sample or the detection of pathogen RNA. Typically, this procedure has been limited to use of gene-specific primers to initiate cDNA synthesis.

The ability to measure the kinetics of a PCR reaction by on-line detection in combination with these reverse transcriptase-PCR techniques has enabled accurate and precise quantitation of RNA copy number with high sensitivity. This has become possible by detecting the reverse transcriptase-PCR product through fluorescence monitoring and measurement of PCR product during the amplification process by fluorescent dual-labeled hybridization probe technologies, such as the 5′ fluorogenic nuclease assay (“TaqMan™”) or endonuclease assay (“CataCleave™”), discussed below.

### Real-Time PCR Using a CataCleave™ Probe

Post amplification amplicon detection is both laborious and time consuming. Real-time methods have been developed to monitor amplification during the PCR process. These methods typically employ fluorescently labeled probes that bind to the newly synthesized DNA or dyes whose fluorescence emission is increased when intercalated into double stranded DNA. Real time detection methodologies are applicable to PCR detection of target nucleic acid sequences in genomic DNA or genomic RNA.

The probes are generally designed so that donor emission is quenched in the absence of target by fluorescence resonance energy transfer (FRET) between two chromophores. The donor chromophore, in its excited state, may transfer energy to an acceptor chromophore when the pair is in close proximity. This transfer is always non-radiative and occurs through dipole-dipole coupling. Any process that sufficiently increases the distance between the chromophores will decrease FRET efficiency such that the donor chromophore emission can be detected radiatively. Common donor chromophores include FAM, TAMRA, VIC, JOE, Cy3, Cy5, and Texas Red.) Acceptor chromophores are chosen so that their excitation spectra overlap with the emission spectrum of the donor. An example of such a pair is FAM-TAMRA. There are also non fluorescent acceptors that will quench a wide range of donors. Other examples of appropriate donor-acceptor FRET pairs will be known to those skilled in the art.

Common examples of FRET probes that can be used for real-time detection of PCR include molecular beacons (e.g., U.S. Pa. No. 5,925,517), TaqMan™ probes (e.g., U.S. Pat. Nos. 5,210,015 and 5,487,972), and CataCleave™ probes (e.g., U.S. Pat. No. 5,763,181). The molecular beacon is a single stranded oligonucleotide designed so that in the unbound state the probe forms a secondary structure where the donor and acceptor chromophores are in close proximity and donor emission is reduced. At the proper reaction temperature the beacon unfolds and specifically binds to the amplicon. Once unfolded the distance between the donor and acceptor chromophores increases such that FRET is reversed and donor emission can be monitored using specialized instrumentation. TaqMan™ and CataCleave™ technologies differ from the molecular beacon in that the FRET probes employed are cleaved such that the donor and acceptor chromophores become sufficiently separated to reverse FRET.

TaqMan™ technology employs a single stranded oligonucleotide probe that is labeled at the 5′ end with a donor chromophore and at the 3′ end with an acceptor chromophore. The DNA polymerase used for amplification must contain a 5′->3′ exonuclease activity. The TaqMan™ probe binds to one strand of the amplicon at the same time that the primer binds. As the DNA polymerase extends the primer the polymerase will eventually encounter the bound TaqMan™ probe. At this time the exonuclease activity of the polymerase will sequentially degrade the TaqMan™ probe starting at the 5′ end. As the probe is digested the mononucleotides comprising the probe are released into the reaction buffer. The donor diffuses away from the acceptor and FRET is reversed. Emission from the donor is monitored to identify probe cleavage. Because of the way TaqMan™ works a specific amplicon can be detected only once for every cycle of PCR. Extension of the primer through the TaqMan™ target site generates a double stranded product that prevents further binding of TaqMan™ probes until the amplicon is denatured in the next PCR cycle.

U.S. Pat. No. 5,763,181, of which content is incorporated herein by reference, describes another real-time detection method (referred to as “CataCleave™”). CataCleave™ technology differs from TaqMan™ in that cleavage of the probe is accomplished by a second enzyme that does not have polymerase activity. The CataCleave™ probe has a sequence within the molecule which is a target of an endonuclease, such as, for example a restriction enzyme or RNAase. In one example, the CataCleave™ probe has a chimeric structure where the 5′ and 3′ ends of the probe are constructed of DNA and the cleavage site contains RNA. The DNA sequence portions of the probe are labeled with a FRET pair either at the ends or internally. The PCR reaction includes a thermostable RNase H enzyme that can specifically cleave the RNA sequence portion of a RNA-DNA duplex. After cleavage, the two halves of the probe dissociate from the target amplicon at the reaction temperature and diffuse into the reaction buffer. As the donor and acceptors separate FRET is reversed in the same way as the TaqMan™ probe and donor emission can be monitored. Cleavage and dissociation regenerates a site for further CataCleave™ binding. In this way it is possible for a single amplicon to serve as a target or multiple rounds of probe cleavage until the primer is extended through the CataCleave™ probe binding site.

### Labeling of a CataCleave™ Probe

The term “probe” comprises a polynucleotide that comprises a specific portion designed to hybridize in a sequence-specific manner with a complementary region of a specific nucleic acid sequence, e.g., a target nucleic acid sequence. In one embodiment, the oligonucleotide probe is in the range of 15-60 nucleotides in length. More preferably, the oligonucleotide probe is in the range of 18-30 nucleotides in length. The precise sequence and length of an oligonucleotide probe of the invention depends in part on the nature of the target polynucleotide to which it binds. The binding location and length may be varied to achieve appropriate annealing and melting properties for a particular embodiment. Guidance for making such design choices can be found in many of the references describing TaqMan™ assays or CataCleave™, described in U.S. Pat. Nos. 5,763,181, 6,787,304, and 7,112,422, of which contents are incorporated herein by reference.

In certain embodiments, the probe is “substantially complementary” to the target nucleic acid sequence.

As used herein, the term “substantially complementary” refers to two nucleic acid strands that are sufficiently complimentary in sequence to anneal and form a stable duplex. The complementarity does not need to be perfect; there may be any number of base pair mismatches, for example, between the two nucleic acids. However, if the number of mismatches is so great that no hybridization can occur under even the least stringent hybridization conditions, the sequence is not a substantially complementary sequence. When two sequences are referred to as “substantially complementary” herein, it means that the sequences are sufficiently complementary to each other to hybridize under the selected reaction conditions. The relationship of nucleic acid complementarity and stringency of hybridization sufficient to achieve specificity is well known in the art. Two substantially complementary strands can be, for example, perfectly complementary or can contain from 1 to many mismatches so long as the hybridization conditions are sufficient to allow, for example discrimination between a pairing sequence and a non-pairing sequence. Accordingly, “substantially complementary” sequences can refer to sequences with base-pair complementarity of 100, 95, 90, 80, 75, 70, 60, 50 percent or less, or any number in between, in a double-stranded region.

As used herein, a “selected region” refers to a polynucleotide sequence of a target DNA or cDNA that anneals with the RNA sequences of a probe. In one embodiment, a “selected region” of a target DNA or cDNA can be from 1, 2, 3, 4, 5, 6, 7, 8, 9, 10, 11, 12, 13, 14, 15, 16, 17, 18, 19, 20, 21, 22, 23, 24, 25 or more nucleotides in length.

As used herein, the site-specific RNase H cleavage refers to the cleavage of the RNA moiety of the Catacleave™ probe that is entirely complimentary to and hybridizes with a target DNA sequence to form an RNA:DNA heteroduplex.

As used herein, “label” or “detectable label” of the CataCleave™ probe refers to any label comprising a fluorochrome compound that is attached to the probe by covalent or non-covalent means.

As used herein, “fluorochrome” refers to a fluorescent compound that emits light upon excitation by light of a shorter wavelength than the light that is emitted. The term “fluorescent donor” or “fluorescence donor” refers to a fluorochrome that emits light that is measured in the assays described in the present invention. More specifically, a fluorescent donor provides energy that is absorbed by a fluorescence acceptor. The term “fluorescent acceptor” or “fluorescence acceptor” refers to either a second fluorochrome or a quenching molecule that absorbs energy emitted from the fluorescence donor. The second fluorochrome absorbs the energy that is emitted from the fluorescence donor and emits light of longer wavelength than the light emitted by the fluorescence donor. The quenching molecule absorbs energy emitted by the fluorescence donor.

Any luminescent molecule, preferably a fluorochrome and/or fluorescent quencher may be used in the practice of this invention, including, for example, Alexa Fluor™ 350, Alexa Fluor™ 430, Alexa Fluor™ 488, Alexa Fluor™ 532, Alexa Fluor™ 546, Alexa Fluor™ 568, Alexa Fluor™ 594, Alexa Fluor™ 633, Alexa Fluor™ 647, Alexa Fluor™ 660, Alexa Fluor™ 680, 7-diethylaminocoumarin-3-carboxylic acid, Fluorescein, Oregon Green 488, Oregon Green 514, Tetramethylrhodamine, Rhodamine X, Texas Red dye, QSY 7, QSY33, Dabcyl, BODIPY FL, BODIPY 630/650, BODIPY 6501665, BODIPYTMR-X, BODIPY TR-X, Dialkylaminocoumarin, Cy5.5, Cy5, Cy3.5, Cy3, DTPA(Eu³⁺)-AMCA and TTHA(Eu³⁺)AMCA.

In one embodiment, the 3′ terminal nucleotide of the oligonucleotide probe is blocked or rendered incapable of extension by a nucleic acid polymerase. Such blocking is conveniently carried out by the attachment of a reporter or quencher molecule to the terminal 3′ position of the probe.

In one embodiment, reporter molecules are fluorescent organic dyes derivatized for attachment to the terminal 3′ or terminal 5′ ends of the probe via a linking moiety. Preferably, quencher molecules are also organic dyes, which may or may not be fluorescent, depending on the embodiment of the invention. For example, in a preferred embodiment of the invention, the quencher molecule is fluorescent. Generally whether the quencher molecule is fluorescent or simply releases the transferred energy from the reporter by non-radiative decay, the absorption band of the quencher should substantially overlap the fluorescent emission band of the reporter molecule. Non-fluorescent quencher molecules that absorb energy from excited reporter molecules, but which do not release the energy radiatively, are referred to in the application as chromogenic molecules.

Exemplary reporter-quencher pairs may be selected from xanthene dyes, including fluoresceins, and rhodamine dyes. Many suitable forms of these compounds are widely available commercially with substituents on their phenyl moieties which can be used as the site for bonding or as the bonding functionality for attachment to an oligonucleotide. Another group of fluorescent compounds are the naphthylamines, having an amino group in the alpha or beta position. Included among such naphthylamino compounds are 1-dimethylaminonaphthyl-5-sulfonate, 1-anilino-8-naphthalene sulfonate and 2-p-touidinyl6-naphthalene sulfonate. Other dyes include 3-phenyl-7-isocyanatocoumarin, acridines, such as 9-isothiocyanatoacridine and acridine orange, N-(p-(2-benzoxazolyl)phenyl)maleimide, benzoxadiazoles, stilbenes, pyrenes, and the like.

In one embodiment, reporter and quencher molecules are selected from fluorescein and rhodamine dyes.

There are many linking moieties and methodologies for attaching reporter or quencher molecules to the 5′ or 3′ termini of oligonucleotides, as exemplified by the following references: Eckstein, editor, Oligonucleotides and Analogues: A Practical Approach (IRL Press, Oxford, 1991); Zuckerman et al., Nucleic Acids Research, 15: 5305-5321 (1987) (3′ thiol group on oligonucleotide); Sharma et al., Nucleic Acids Research, 19: 3019 (1991) (3′ sulfhydryl); Giusti et al., PCR Methods and Applications, 2: 223-227 (1993) and Fung et al., U.S. Pat. No. 4,757,141 (5′ phosphoamino group via Aminolink™ II available from Applied Biosystems, Foster City, Calif.) Stabinsky, U.S. Pat. No. 4,739,044 (3′ aminoalkylphosphoryl group); Agrawal et al., Tetrahedron Letters, 31: 1543-1546 (1990) (attachment via phosphoramidate linkages); Sproat et al., Nucleic Acids Research, 15: 4837 (1987) (5′ mercapto group); Nelson et al., Nucleic Acids Research, 17: 7187-7194 (1989) (3′ amino group); and the like.

Rhodamine and fluorescein dyes are also conveniently attached to the 5′ hydroxyl of an oligonucleotide at the conclusion of solid phase synthesis by way of dyes derivatized with a phosphoramidite moiety, e.g., Woo et al., U.S. Pat. No. 5,231,191; and Hobbs, Jr., U.S. Pat. No. 4,997,928.

### Attachment of a CataCleave™ Probe to a Solid Support

In one embodiment, the oligonucleotide probe can be attached to a solid support. Different probes may be attached to the solid support and may be used to simultaneously detect different target sequences in a sample. Reporter molecules having different fluorescence wavelengths can be used on the different probes, thus enabling hybridization to the different probes to be separately detected.

Examples of preferred types of solid supports for immobilization of the oligonucleotide probe include controlled pore glass, glass plates, polystyrene, avidin coated polystyrene beads cellulose, nylon, acrylamide gel and activated dextran, controlled pore glass (CPG), glass plates and high cross-linked polystyrene. These solid supports are preferred for hybridization and diagnostic studies because of their chemical stability, ease of functionalization and well defined surface area. Solid supports such as controlled pore glass (500 \acute{Å}, 1000 \acute{Å}) and non-swelling high cross-linked polystyrene (1000 \acute{Å}) are particularly preferred in view of their compatibility with oligonucleotide synthesis.

The oligonucleotide probe may be attached to the solid support in a variety of manners. For example, the probe may be attached to the solid support by attachment of the 3′ or 5′ terminal nucleotide of the probe to the solid support. However, the probe may be attached to the solid support by a linker which serves to distance the probe from the solid support. The linker is most preferably at least 6 atoms in length.

Hybridization of a probe immobilized to a solid support generally requires that the probe be separated from the solid support by at least 30 atoms. In order to achieve this separation, the linker may include a spacer positioned between the linker and the 3′ nucleoside. For oligonucleotide synthesis, the linker arm is usually attached to the 3′-OH of the 3′ nucleoside by an ester linkage which can be cleaved with basic reagents to free the oligonucleotide from the solid support.

A wide variety of linkers are known in the art which may be used to attach the oligonucleotide probe to the solid support. The linker may be formed of any compound which does not significantly interfere with the hybridization of the target sequence to the probe attached to the solid support. The linker may be formed of a homopolymeric oligonucleotide which can be readily added on to the linker by automated synthesis. Alternatively, polymers such as functionalized polyethylene glycol can be used as the linker. Such polymers are preferred over homopolymeric oligonucleotides because they do not significantly interfere with the hybridization of probe to the target oligonucleotide. Polyethylene glycol is particularly preferred because it is commercially available, soluble in both organic and aqueous media, easy to functionalize, and completely stable under oligonucleotide synthesis and post-synthesis conditions.

The linkages between the solid support, the linker and the probe are preferably not cleaved during removal of base protecting groups under basic conditions at high temperature. Examples of preferred linkages include carbamate and amide linkages. Immobilization of a probe is well known in the art and one skilled in the art may determine the immobilization conditions.

According to one embodiment of the method, the CataCleave™ probe is immobilized on a solid support. The CataCleave™ probe comprises a detectable label and DNA and RNA nucleic acid sequences, wherein the probe's RNA nucleic acid sequences are entirely complementary to a selected region of the target DNA sequence and the probe's DNA nucleic acid sequences are substantially complementary to DNA sequences adjacent to the selected region of the target DNA sequence. The probe is then contacted with a sample of nucleic acids in the presence of RNase H and under conditions where the RNA sequences within the probe can form a RNA:DNA heteroduplex with the complementary DNA sequences in the PCR fragment. RNase H cleavage of the RNA sequences within the RNA:DNA heteroduplex results in a real-time increase in the emission of a signal from the label on the probe, wherein the increase in signal indicates the presence of the target DNA sequence.

According to another embodiment of the method, the CataCleave™ probe, immobilized on a solid support, comprises a detectable label and DNA and RNA nucleic acid sequences, wherein the probe's RNA nucleic acid sequences are entirely complementary to a selected region of the target DNA sequence and the probe's DNA nucleic acid sequences are substantially complementary to DNA sequences adjacent to the selected region of the target DNA sequence. The probe is then contacted with a sample of nucleic acids in the presence of RNase H and under conditions where the RNA sequences within the probe can form a RNA:DNA heteroduplex with the complementary DNA sequences in the PCR fragment. RNase H cleavage of the RNA sequences within the RNA:DNA heteroduplex results in a real-time increase in the emission of a signal from the label on the probe.

Immobilization of the probe to the solid support enables the target sequence hybridized to the probe to be readily isolated from the sample. In later steps, the isolated target sequence may be separated from the solid support and processed (e.g., purified, amplified) according to methods well known in the art depending on the particular needs of the researcher.

### Kits

The disclosure herein also provides for a kit format which comprises a package unit having one or more reagents for the hot start amplification of a target nucleic acid. The kit may also contain one or more of the following items: buffers, instructions, and positive or negative controls. Kits may include containers of reagents mixed together in suitable proportions for performing the methods described herein. Reagent containers preferably contain reagents in unit quantities that obviate measuring steps when performing the subject methods.

Kits may also contain reagents for real-time PCR including, but not limited to, a hot start composition comprising a thermostable nucleic acid polymerase, hot start thermostable RNase H, primers selected to amplify a target nucleic acid sequence, and a labeled CataCleave™ oligonucleotide probe that anneals to the real-time PCR product and allows for the quantitative detection of the target nucleic acid sequence according to the methodology described herein.

In another embodiment, the kit reagents further comprised reagents for the extraction of genomic DNA or RNA from a biological sample. Kit reagents may also include reagents for reverse transcriptase-PCR analysis where applicable.

Any patent, patent application, publication, or other disclosure material identified in the specification is hereby incorporated by reference herein in its entirety. Any material, or portion thereof, that is the to be incorporated by reference herein, but which conflicts with existing definitions, statements, or other disclosure material set forth herein is only incorporated to the extent that no conflict arises between that incorporated material and the present disclosure material.

### EXAMPLES

The following examples set forth methods for using the hot start composition according to the present invention. It is understood that the steps of the methods described in these examples are not intended to be limiting. Further objectives and advantages of the present invention other than those set forth above will become apparent from the examples which are not intended to limit the scope of the present invention.

A PCR reaction contains forward and reverse primers, a dual-labeled DNA-RNA-DNA probe, a polymerase with 5′ exonuclease activity, and RNase H. As the polymerase extends the primers and synthesizes the nascent strand, the 5′ to 3′ exonuclease activity of the polymerase degrades the probe that has annealed to the template. In the meantime, due to the chimeric structure of the probe, once bound to the target sequence, the probe itself also serves as a substrate of RNase H. Therefore, the supplementary endonuclease activity accelerates the probe degradation kinetics, resulting in an elevated signal boost when compared to endonuclease (TaqMan®) alone.

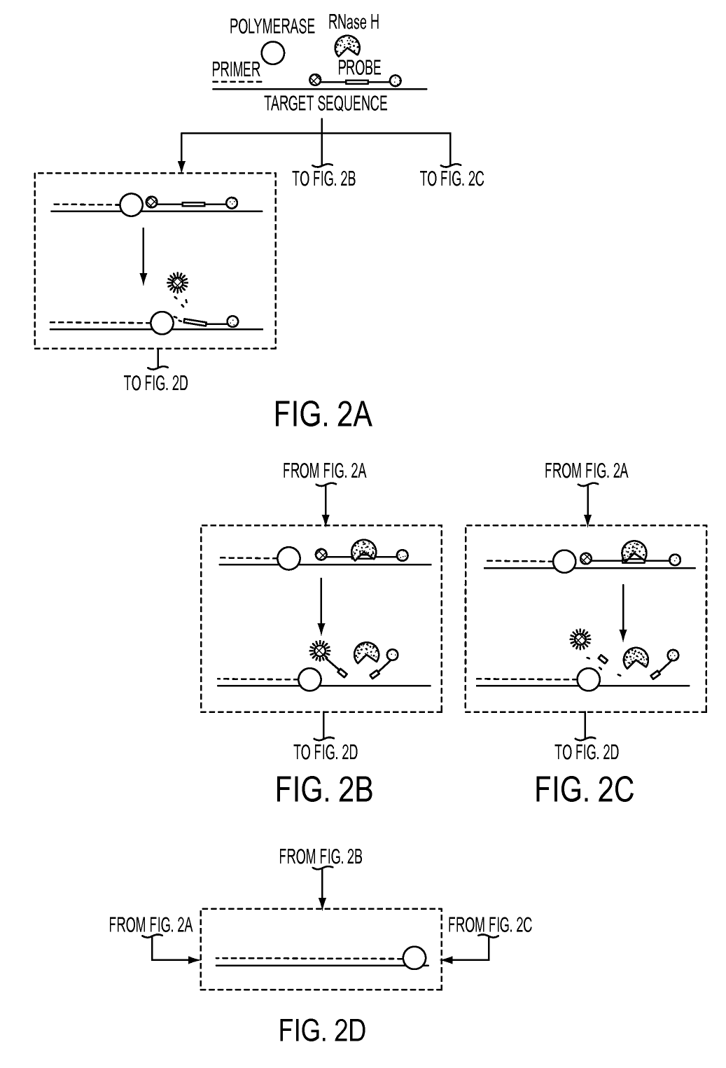

FIG. 2 illustrates several schemes of possible probe cleavage mechanisms in the presence of both endonuclease and exonuclease. A typical PCR reaction contains forward and reverse primers, polymerase, RNase H, probe and a target sequence. During PCR cycling, probe can be cleaved by the polymerase's 5′-3′ exonuclease activity during primer extension (scheme A), or endonuclease activity by RNase H (scheme B), or by both polymerase and RNase H (scheme C). At the end of each PCR cycle, the nascent complementary strand is synthesized and, therefore, the probe binding site is occluded (scheme D). In a typical TaqMan® reaction, however, probe degradation is entirely attributed to polymerase's 5′-3′ exonuclease (scheme A).

### Example 1

In the present study, cleavage of a HBV probe was tested in the presence and absence of RNase HII. Profiles of probe degradation under these conditions were analyzed and compared.

Materials and Methods

Instrument: Roche LightCycler 480 II instrument.

Test Protocol

Real-time PCR reactions were carried out with or without RNase HII. Briefly, two sets of reaction master mixes were prepared. Master Mix (or Condition) “I” contains all components for PCR/CataCleave™, and Master Mix (or Condition) “II” contains all except RNase HII. SYBR Green I was added to monitor PCR performance. Each 25 μL PCR reaction contains 1×PCR Buffer (Life Tech), 2.5 U Platinum Taq polymerase (Life Tech), 0.4 U uracil-DNA-glycosylase (Life Tech), 1 μL SYBR Green I dye, 48 nM forward primer HBV F5d (CTC GTG TTA CAG GCG GGG TTT TTC TTG TTG ACA A (SEQ ID NO: 1)), 48 nM reverse primer HBV R5b (AAC GCC GCA GAC ACA TCC AGC GA (SEQ ID NO: 2)), 80 nM probe HBV P1 (5′-Cal Fluor 610-TGG CCA AAA TTC rGrCrA rGTC CCC CAA CCT CCA AT-3′ BHQ2 (SEQ ID NO: 3)), 200 μM dATP/UTP/CTP/GTP, 100 μM dTTP, with or without 2.5 U RNase HII, and 2 μL of DNA template (HBV genotype B plasmid). RNA sequences are denoted as rR in the sequence listing. The following template concentrations in duplicates were tested, 10⁶ and 10² copies per reaction. Water was used for blank (or negative) controls. The real-time PCR reactions began with 95° C. for 5 min, then 50 cycles of 95° C. for 10 sec, 55° C. for 10 sec and 65° C. for 30 seconds, followed by a melting curve analysis from 37° C. to 97° C. Color compensation of SYBR Green I and Cal Fluor Red 610 dyes were performed prior to data analysis.

Results and Discussion

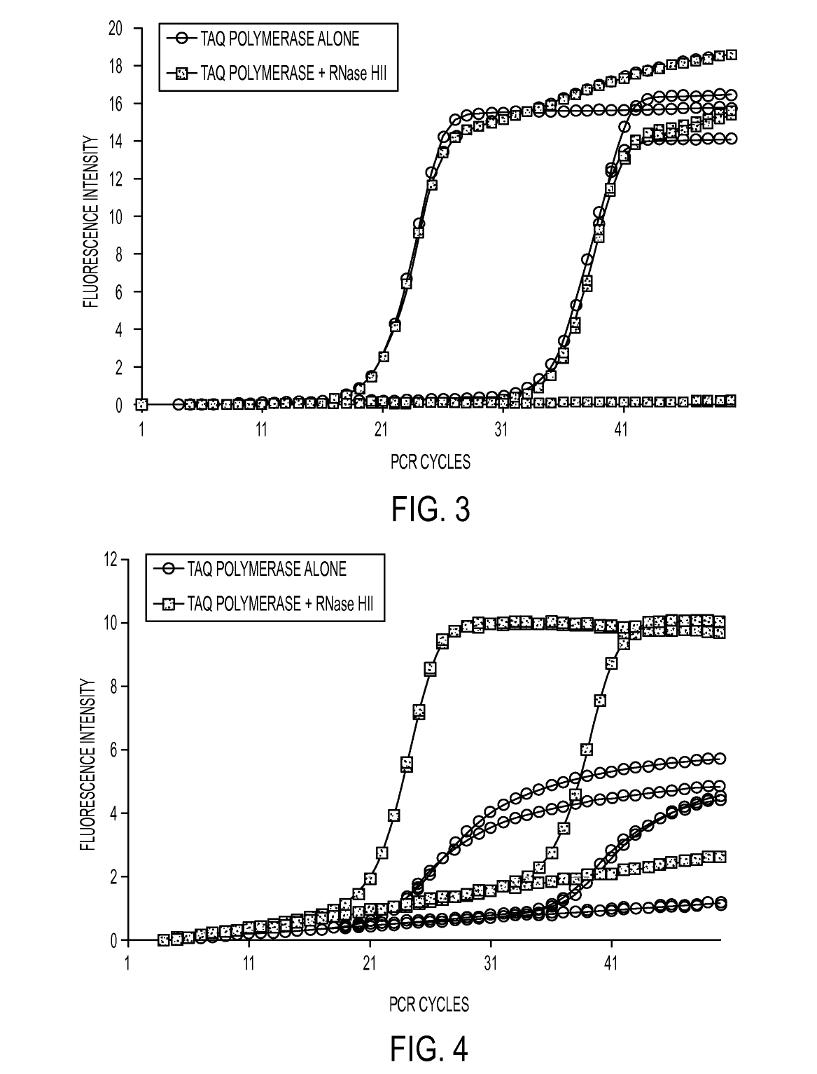

FIG. 3 is a graph showing performance of PCR in the presence and absence of RNase H. A non-specific DNA intercalating dye, SYBR Green I, was used to monitor amplification of the target sequence. It was demonstrated that inclusion of RNase H does not change the exponential amplification performance of the reaction. Since the probe was labeled with Cal Fluor 610, cleavage kinetics therefore could be monitored separately. In FIG. 4, the Fluorescence signal increase observed in the negative controls may be attributed to probe degradation caused by hydrolysis. On the other hand, the fluorescence signal increase observed in the positive controls with RNase HII may be attributed to probe degradation and probe cleavage by Taq polymerase and RNase HII. Without RNase HII, the fluorescence increase may be attributed to probe degradation and the exonuclease activity of Taq polymerase. Due to fluorescence fluctuation during the first few cycles, fluorescence readings used in the present comparison started from cycle 5.

The fluorescent increases from cycle 5 to cycle 50 performed on two different target concentrations of HBV in duplicate (named HBV 1e6_{—}1 (10⁶ copies/reaction), HBV 1e6_{—}2 (10⁶ copies/reaction), HBV 1e2_{—}1 (10² copies/reaction), and HBV 1e2_{—}2 (10² copies/reaction) were measured with regard to template concentration.

Results are shown in Table 1 below. The net fluorescence increase contributed by RNase HII and Taq polymerase was 7.17 while the increase was 3.70 when only Taq polymerase was used. Therefore, an overall net fluorescence increase of 3.47 (or 93.8%) was achieved when RNase H was present in addition to Taq polymerase.

TABLE 1
Fluorescence increase from cycle 5 to cycle 50.

| | | | | Average | ΔΔRₚₒₛᵢₜᵢᵥₑ − |
|---|---|---|---|---|---|
| Template | ΔR₅ | ΔR₅₀ | Δ(ΔR₅₀ − ΔR₅) | ΔΔR | ΔΔR_{negative} |
| | Taq polymerase + RNase HII | | | | |
| HBV 1e6_1 | 0.06 | 9.66 | 9.60 | 9.76 | 7.17 |
| HBV 1e6_2 | 0.05 | 9.74 | 9.69 | | |
| HBV 1e2_1 | 0.05 | 10.06 | 10.01 | | |
| HBV 1e2_2 | 0.05 | 9.78 | 9.73 | | |
| Blank_1 | 0.05 | 2.63 | 2.58 | 2.59 | |
| Blank_2 | 0.06 | 2.66 | 2.59 | | |
| | Taq polymerase only | | | | |
| HBV 1e6_1 | 0.02 | 4.85 | 4.83 | 4.86 | 3.70 |
| HBV 1e6_2 | 0.03 | 5.70 | 5.68 | | |
| HBV 1e2_1 | 0.03 | 4.43 | 4.40 | | |
| HBV 1e2_2 | 0.02 | 4.55 | 4.53 | | |
| Blank_1 | 0.03 | 1.20 | 1.17 | 1.15 | |
| Blank_2 | 0.02 | 1.16 | 1.14 | | |

### Example 2

Cleavage profiles of a Salmonella probe in the presence and absence of RNase HII were examined. The Salmonella CataCleave probe, named inv-CC-Probe 1 contains the following nucleotides (RNA bases are denoted as rR in the sequence listing.)

5′-/FAM/TCT GGT TGA rUrUrU rCCT GAT CGC A/31ABkFQ/-3′ (SEQ ID NO: 4).

Experimental Methods

The TaqMan® and CataCleave™ assays were conducted using a serial dilution series of plasmid DNA containing the Salmonella invA target. The dilution series consisted of seven 10-fold serial dilutions from 10⁵ copies of plasmid down to 1 copy of plasmid (6 log range), as well as a negative control. The PCR reactions were set up in the same PCR plate and run under the same cycling conditions for both assays. Each 25 μL PCR reaction contained 1×ICAN Buffer, 4 mM MgCl₂, 80 μM dNTP and 160 μM dUTP mix, 800 nM Salmonella-F1 forward primer, 800 nM Sal-InvR2 reverse primer, 1 U uracil-DNA-glycosylase, 2.5 U Taq DNA Polymerase, 200 nM inv-CC-Probel CataCleave probe, with or without 2.5 U RNase H II, and water to 25 μL. The real-time PCR reactions began with 37° C. for 10 min, 95° C. for 10 min, followed by 50 cycles of 95° C. for 15 sec, 55° C. for 15 sec and 72° C. for 20 sec.

Results

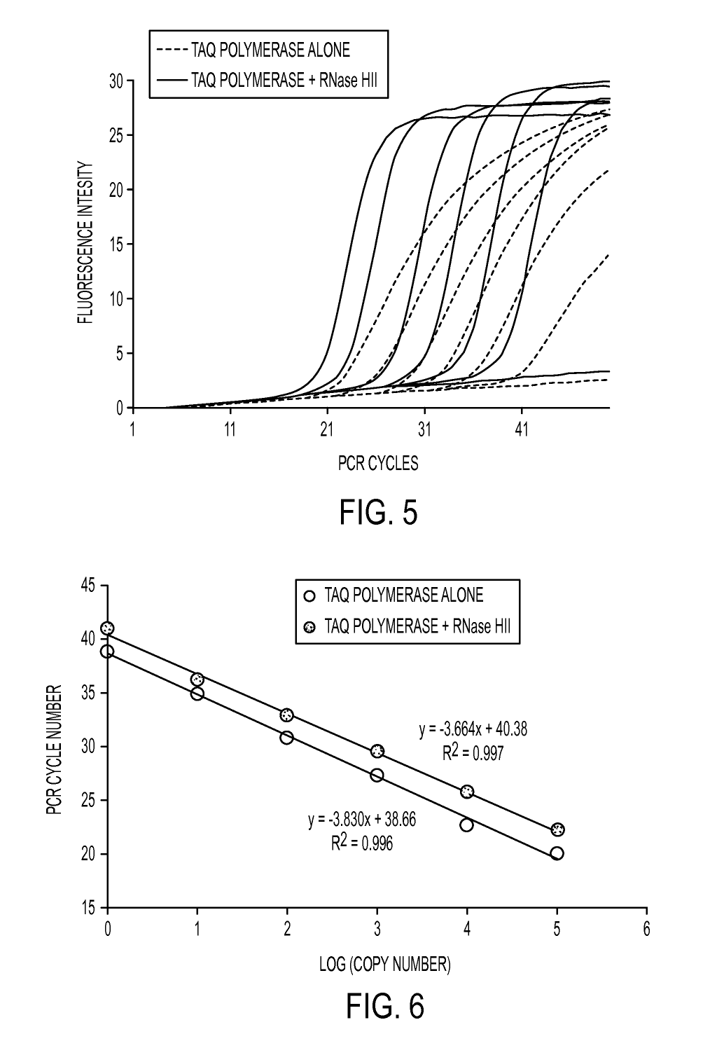

The amplification curves for the TaqMan® and CataCleave™ real time PCR (qPCR) reactions are shown in FIG. 5. Under either condition it was possible to detect the full dilution series from 10⁵ copies to as little as 1 copy of plasmid target. However, when RNase HII was not included in the reactions, the endpoint fluorescence was significantly lower when detecting low concentrations of template (e.g., 10 copies and 1 copy) compared to the detection of 10⁵ copies. In contrast, when RNase HII was present in the reactions, the endpoint fluorescence was approximately the same for all concentrations of target. Furthermore, addition of RNase HII, in comparison to the Taqman reactions, consistently achieved decreased Cp values by approximately 2 cycles at each dilution level.

### CONCLUSIONS

These results highlight an advantage that inclusion of RNase HII improves uniformity in fluorescent signal intensities, especially at low template concentrations. It was revealed that, while the endpoint fluorescence for 1 copy is lower than 10⁵ copies in the TaqMan® qPCR assay, the endpoint fluorescence for all concentrations is the same in the CataCleave™ qPCR assay. Despite a slight increase in background fluorescence due to non-specific enzymatic probe cleavage, the overall gain in signal-to-noise ratio by RNase HII is significantly higher and independent of the template concentration, as compared to TaqMan®.

### Example 3

Data from the experiment described in Example 2 were also analyzed to determine if the addition of RNase HII had any impact on the ability of the Light Cycler 480 II software to automatically assign crossing point values to the data. The Crossing point (Cp) is determined by analyzing the second derivative of the fluorescence intensity data. The data are shown in the Table 2 below.

TABLE 2
Crossing point analysis of TaqMan and CataCleave data.

Salmonella

| Copy # | Log | Taqman | CataCleave |
|---|---|---|---|
| 0 | N/A | Negative | Negative |
| 1 | 0 | 38.84 | 40.92 |
| 10 | 1 | 34.84 | 36.17 |
| 100 | 2 | 30.86 | 32.83 |
| 1000 | 3 | 27.26 | 29.49 |
| 10000 | 4 | 22.67 | 25.71 |
| 100000 | 5 | 20.05 | 22.21 |

| | | |
|---|---|---|
| Slope | −3.83 | −3.66 |
| PCR efficiency | 0.82 | 0.87 |
| R² | 0.997 | 0.997 |

The data were also graphed as shown in FIG. 6. PCR efficiency is a measure of the deviation of amplification efficiency from the theoretical optimum of 3.333 cycles for each 10 fold change in template concentration (2^{3.333}=10) when the target is amplified exponentially. Reactions containing Taq polymerase, CataCleave™ probe, and RNase HII had a PCR efficiency of 87%, while the reactions containing Taq polymerase and TaqMan probe had an efficiency of 82%. Furthermore, addition of RNase HII consistently achieved decreased Cp values by approximately 2 cycles at each dilution level. For example, the difference in Cp between TaqMan® and CataCleave™ for 1000 input copies was almost 2 cycles, or a 4 fold underestimation of the actual initial template concentration in the sample. This could influence quantification of nucleic acid templates in PCR reactions, and have consequences with regards to initiating, terminating, or modifying a drug treatment regimen.

### Example 4

In this example the probe degradation kinetics of CataCleave™ and TaqMan® with a single-stranded DNA template (200 nucleotides long) were investigated. In a real-time PCR/CataCleave™ reaction, the kinetics of probe cleavage are complex, due to the exponential increase in template concentration and the simultaneous occurrence of CataCleave™ and PCR. Probe cleavage by either Taq exonuclease activity, RNase HII endonuclease activity, or both was difficult to monitor separately. As a simple and straightforward alternative, CataCleave™ and TaqMan® reactions were characterized separately using a long single stranded DNA template.

The single-stranded DNA template, the sequence of which is listed as SEQ ID NO: 6 was synthesized by IDT and is a 200-nt antisense strand of the HBV amplicon generated by the PCR described in Example 1 (equivalent to 94% of the amplicon size). It harbors full annealing sites for the forward primer and the probe, and a partial sequence of the reverse primer. Using this template, the kinetics of CataCleave™ probe degradation could be simulated in the absence of interference from exponential target amplification. When measuring TaqMan® activity, only forward primer, Taq polymerase, and the single-stranded DNA template were used. Assuming 100% reaction efficiency, one probe molecule is cleaved by 5′ exonuclease activity during one round of primer extension per DNA template molecule. Due to the lack of a reverse primer and that the synthesized nascent strand has no binding site for either the forward primer or the probe, no PCR amplification occurs and the number of copies of the single-stranded DNA template remains constant. Therefore, provided with a constant amount of probe binding target, this gives an insight into probe cleavage kinetics due to either exonuclease or endonuclease or both under typical PCR temperature cycling conditions.

Materials and Methods, and Instrument List

| Instrument | Manufacturer |
|---|---|
| LightCycler 480 II | Roche Diagnostics |

### Reagent List

| Reagent | Manufacturer |
|---|---|
| Platinum Taq polymerase | Life Tech |
| RNase HII | Samsung |
| UDG | Life Tech |
| HBV F5d forward primer | CTC GTG TTA CAG GCG GGG TTT TTC |
| | TTG TTG ACA A (SEQ ID NO: 1) |
| HBV (Cal Fluor610) probe | 5′-Cal Fluor 610-TGG CCA AAA TTC |
| | rGrCrA rGTC CCC CAA CCT CCA AT-3′ |
| | BHQ2 (SEQ ID NO: 2) |
| SYBR Green I dye | Molecular Probes |
| HBV target | 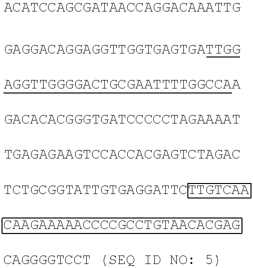 |
| | Underlined region: forward primer binding site |
| | Boxed region: probe binding site |
| dATP | Life Tech |
| dGTP | Life Tech |
| dCTP | Life Tech |
| dTTP | Life Tech |
| dUTP | Life Tech |

Degradation of HBV CataCleave™ Probe by Taq Polymerase or RNase HII

The probe target is a single-stranded 200-mer DNA sequence containing one annealing site for the HBV forward primer, and another annealing site for the HBV probe.

The single-stranded target DNA was provided as the reaction template. In addition, only forward primer but no reverse primer was used to initiate primer extension, therefore the reaction proceeded without exponential amplification. Under such conditions, only one round of TaqMan® probe cleavage and one or more rounds of CataCleave™ probe cleavage would occur per cycle. Due to the fact the template was almost the same size as the amplicon whereas it was single-stranded, CataCleave™ probe cleavage would occur without aid of template amplification. Therefore, the current system can separate TaqManO and CataCleave™ activities apart.

Three reaction conditions were tested, in the presence Taq and the presence or absence of RNase HII and a negative control. A 25-uL reaction solution contained 5 uL of Buffer 15, 0.25 uL of dACGU/TTP (10/5 mM), 48 nM HBV-F5d forward primer (SEQ ID NO: 1), 200 nM HBV probe (SEQ ID NO: 3), 0.8 nM HBV probe target (SEQ ID NO: 5), and the following components:
1) Condition I: 2.5 units of Platinum Taq® polymerase
2) Condition II: 2.5 units of Platinum Taq® polymerase and 2.5 units of RNase HII,
3) Condition III: No Taq polymerase or RNase HII, and water to 25 uL.

The primer extension reaction followed the same HBV PCR cycling parameters. It began with 95° C. for 5 min, followed by 90 cycles of 95° C. for 10 sec, 55° C. for 10 sec, and 65° C. for 30 sec.

Results

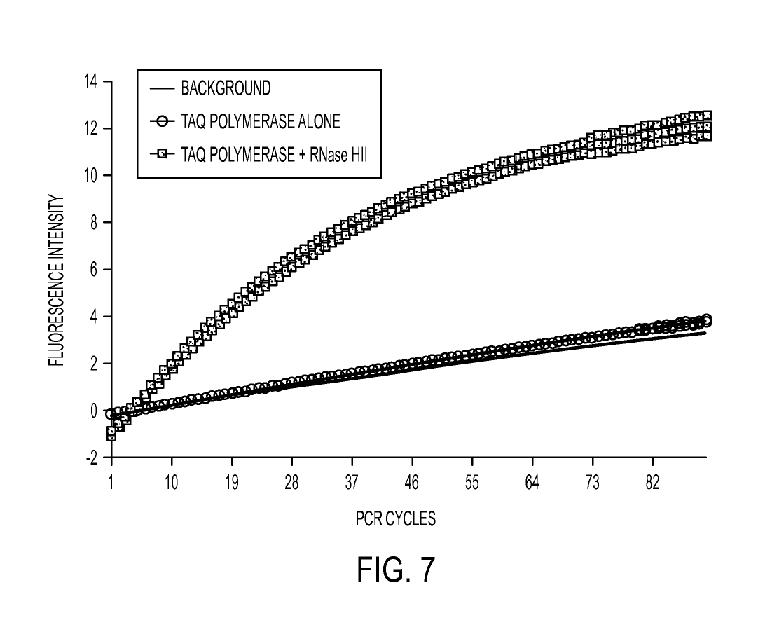

During the first 9 cycles of amplification, the fluorescence increase in reactions containing Taq polymerase alone was 0.39, while in reactions containing both Taq polymerase and RNase HII the fluorescence increase was 2.55. During the same time the increase in fluorescence intensity for the background reaction, a nuclease-free control, was 0.29 (FIG. 7). Therefore, after background correction, the net fluorescence intensity increase was 0.1 in reactions containing only Taq polymerase, and 2.24 in reactions containing both Taq polymerase and RNase HII. Results are shown in Table 3-A and 3-B.

TABLE 3
Fluorescence increase during the first 9 cycles. Each condition was tested with four replicates.

| 3−A. | | | | | 3−B. | | | | | | | |
|---|---|---|---|---|---|---|---|---|---|---|---|---|
| | Taq pol only | | | | Taq pol + RNase HII | | | | Background | | | |
| PCR Cycle | 1 | 2 | 3 | 4 | 1 | 2 | 3 | 4 | 1 | 2 | 3 | 4 |
| 1 | −0.13 | −0.13 | −0.15 | −0.14 | −1.01 | −1.05 | −0.91 | −0.91 | −0.06 | −0.07 | −0.07 | −0.07 |
| 2 | −0.09 | −0.09 | −0.10 | −0.09 | −0.66 | −0.70 | −0.61 | −0.60 | −0.06 | −0.07 | −0.07 | −0.07 |
| 3 | −0.05 | −0.04 | −0.06 | −0.05 | −0.33 | −0.35 | −0.31 | −0.31 | −0.04 | −0.05 | −0.04 | −0.05 |
| 4 | −0.01 | 0.00 | 0.00 | −0.01 | −0.01 | 0.00 | 0.00 | 0.00 | −0.01 | 0.00 | −0.01 | −0.01 |
| 5 | 0.05 | 0.05 | 0.06 | 0.06 | 0.33 | 0.35 | 0.31 | 0.31 | 0.03 | 0.05 | 0.04 | 0.05 |
| 6 | 0.09 | 0.09 | 0.10 | 0.10 | 0.66 | 0.69 | 0.61 | 0.60 | 0.09 | 0.08 | 0.08 | 0.09 |
| 7 | 0.14 | 0.14 | 0.15 | 0.14 | 0.98 | 1.03 | 0.93 | 0.91 | 0.13 | 0.13 | 0.12 | 0.14 |
| 8 | 0.19 | 0.19 | 0.19 | 0.20 | 1.30 | 1.36 | 1.22 | 1.20 | 0.17 | 0.17 | 0.18 | 0.18 |
| 9 | 0.24 | 0.25 | 0.26 | 0.25 | 1.62 | 1.68 | 1.51 | 1.50 | 0.21 | 0.22 | 0.23 | 0.24 |
| Net Rn increase | 0.36 | 0.37 | 0.42 | 0.39 | 2.63 | 2.74 | 2.42 | 2.42 | 0.27 | 0.29 | 0.30 | 0.31 |

| | | | |
|---|---|---|---|
| Average et Rn | 0.39 | 2.55 | 0.29 |

In this experiment, 24 units of net fluorescence increase represented a total of 3×10¹² probe molecules (1.25×10¹¹ probe molecules per fluorescent unit). There were 9×10⁹ molecules of single-stranded probe-binding target in the system. Therefore, Taq polymerase alone promoted the cleavage of 1.39 probe molecules per cycle per target molecule. The combination of Taq polymerase and RNase HII promoted the cleavage of 35.42 probe molecules per cycle per target molecule. The results are shown in FIG. 7, which shows a comparison of probe cleavage by Taq polymerase, and by both Taq polymerase and RNase H. Probe cleavage due to hydrolysis was used as a background control.

The experimental results described above show that the rate of probe cleavage due to the 5′ exonuclease activity of Taq polymerase alone was significantly lower than in an identical reaction containing both Taq polymerase and RNase H. The results of this experiment indicate that, when the amount of the probe-binding target remains constant, the rate of probe cleavage due to endonuclease digestion by RNase HII is much higher than the contribution by the exonuclease activity of Taq polymerase during primer extension. This may connect to the observation of uniform end-point fluorescence intensities in CataCleave reactions, in comparison to heterogeneous end-point fluorescence intensities observed in Taqman reactions. Of more importance is the increase in the signal to noise ratio in reactions containing both components as compared to a TaqMan reaction alone. This is especially apparent in a situation where the starting template concentration is limited and the resulting TaqMan signal plateau is low.

1. A method of detecting a target sequence in a sample, the method comprising:
(a) amplifying the target sequence in the sample to produce an increased number of copies of the target sequence, the amplifying including hybridizing a first target-sequence specific primer and a second target-sequence specific primer to the target nucleic acid in the sample to obtain a hybridized product of the target nucleic acid and the primers, and extending the first and the second primers of the hybridized product with a template-dependent nucleic acid polymerase to produce an extended primer product;
(b) hybridizing the extended primer product to at least one probe oligonucleotide to obtain a hybridized product of the extended primer product:probe oligonucleotide, wherein the probe comprises a 5′-DNA sequence and an RNA sequence, wherein the probe is coupled to a detectable label, said label being a fluorescence resonance energy transfer (FRET) pair, wherein the FRET pair emits a FRET signal, one of said pair is coupled to the 3′-end of the probe and the other of said pair is coupled to the 5′-end of the probe, and wherein said probe does not hybridize to either said first or said second primer;
(c) subjecting said hybridized product of the extended primer product:probe oligonucleotide to an RNase H activity and to an exonuclease activity;
(d) detecting an emission of a signal from said label measuring the FRET signal emitted by one of the FRET pair.

2. (canceled)

3. The method according to claim 1, wherein the amplifying is performed by a polymerase chain reaction.

4. The method according to claim 1, wherein the amplifying, the hybridizing and the contacting are simultaneously or sequentially carried out.

5. The method according to claim 1, further comprising cultivating the sample containing the target sequence in an enriched medium before the amplifying, to enhance growth of a pathogen containing the target sequence.

6. The method according to claim 1, wherein the target sequence is an RNA.

7. A method according to claim 6, wherein the method further comprises a step of producing a complementary DNA sequence of the target RNA sequence.

8. The method according to claim 1, wherein the target sequence is a DNA.

9. The method according to claim 1, wherein the exonuclease activity is originated from the DNA polymerase.

10. The method according to claim 1, wherein the RNase H is RNase HII.

11. The method according to claim 1, wherein the RNase H is thermostable.

12. The method according to claim 1, which comprises plural cycles of steps (a) to (d), and wherein the amplification of said target sequence during said plurality of cycles exhibits an exponential phase, a linear phase, and a plateau phase,
and wherein the signal intensity measured at the plateau phase is higher by 1% or more compared to the signal intensity measured at the plateau phase of the same method performed in the absence of the RNase H.

13. The method according to claim 1, wherein the probe has a structure of 5′-DNA-RNA-DNA structure.

14. The method according to claim 12, wherein the RNA sequence has from 1 to 10 nucleotides.


SEQUENCE LISTING (5 sequences)

SEQ ID NO: 1 - DNA, 34 bp, Artificial Sequence
  Description of Artificial Sequence Synthetic oligonucleotide
ctcgtgttacaggcggggtttttcttgttgacaa

SEQ ID NO: 2 - DNA, 23 bp, Artificial Sequence
  Description of Artificial Sequence Synthetic oligonucleotide
aacgccgcagacacatccagcga

SEQ ID NO: 3 - DNA, 32 bp, Artificial Sequence
  Description of Artificial Sequence Synthetic oligonucleotide
tggccaaaattcgcagtcccccaacctccaat

SEQ ID NO: 4 - DNA, 22 bp, Artificial Sequence
  Description of Artificial Sequence Synthetic probe
tctggttgauuucctgatcgca

SEQ ID NO: 5 - DNA, 200 bp, Unknown
  Description of Unknown HBV probe target polynucleotide
acatccagcgataaccaggacaaattggaggacaggaggttggtgagtgattggaggttggggactgcgaattttggccaagacacacgggtgatccccctagaaaattgagagaagtccaccacgagtctagactctgcggtattgtgaggattcttgtcaacaagaaaaaccccgcctgtaacacgagcaggggtcct